(19) United States
(12) Patent Application Publication
(10) Pub. No.: US 2018/0311505 A1
(43) Pub. Date: Nov. 1, 2018

(54) METHODS FOR TREATING CANCER BY ENHANCING INTRATUMORAL IMMUNE RESPONSE

(71) Applicant: THE GENERAL HOSPITAL CORPORATION, Boston, MA (US)

(72) Inventors: David E. FISHER, Newton, MA (US); Jennifer A. LO, Center Cambridge, MA (US); Dieter MANSTEIN, Charlestown, MA (US); Masayoshi KAWAKUBO, Malden, MA (US)

(73) Assignee: THE GENERAL HOSPITAL CORPORATION, Boston, MA (US)

(21) Appl. No.: 15/773,919

(22) PCT Filed: Nov. 3, 2016
(86) PCT No.: PCT/US2016/060321

§ 371 Date: May 4, 2018

Related U.S. Application Data
(60) Provisional application No. 62/251,336, filed on Nov. 5, 2015.

(51) Int. Cl. A61N 5/06; A61P 35/00; C07K 16/28; A61K 31/4745; A61N 1/40

(52) U.S. Cl. CPC A61N 5/062; A61P 35/00; C07K 16/2818; A61N 2005/067; A61N 5/0616; A61N 1/40; A61K 31/4745

(57) ABSTRACT

Provided herein are methods that can be used to produce a local immune response in cancer tissue and/or enhance effectiveness of cancer treatment in a subject through application of one or more combinations of: an ablative fractional laser procedure, a checkpoint inhibitor, and an endosomal TLR agonist (e.g., a TLR3, TLR7, TLR8 or TLR9 agonist).

### FIELD OF THE DISCLOSURE

The present disclosure relates to the treatment of tumors in a subject.

### BACKGROUND

In immune surveillance, altered proteins are distinct from self proteins and are not protected by central tolerance. These “neoantigens” can potentially be recognized by the immune system. As but one example, the mutational burden associated with ultraviolet radiation (UVR) translates to an abundance of neoantigens in melanoma.

The importance of immune responses in cancer, including melanoma, has long been appreciated, with reports of spontaneous regression of metastatic melanomas first published 60 years ago^{6,7}. Immunosuppressed individuals are at greater risk of melanoma⁸ and prolonged disease dormancy followed by “ultra-late” recurrences is observed in some patients⁹. Early discovery of immune infiltrates and tumor-specific antibodies as positive prognostic factors in melanoma provided additional evidence of tumor interaction with the immune system^{10,11}. The high immunogenicity of melanoma may reflect the preponderance of UV-induced neoantigens that can serve as targets of immune responses.

Fractional tissue treatment is a fairly recent development that generally involves formation of small, spatially-separated regions of damage in tissue. The damaged regions are small, typically having a dimension that is about 1 mm or less. Such damage regions can be generated in tissue using various modalities, including irradiation by a laser or other optical energy, focused ultrasound, administration of radiofrequency (RF) energy via spaced-apart electrodes, etc. Typically the amount of damage induced is between about 5% and 50% as measured, e.g., in a surface or projected area of the tissue being treated, with areas or volumes of tissue between the damage regions remaining relatively unaffected. Generating damage in such spatially-separated small regions has been observed to be well-tolerated and to induce a healing response that can, for example, rejuvenate skin tissue with little risk of infection.

Non-ablative fractional processes generally refer to processes in which the small regions of tissue are damaged (typically by localized heating) without removal of tissue. Ablative fractional treatment generally refers to processes in which some amount of tissue is removed, e.g., by energy-induced vaporization or mechanical extraction. Ablative fractional processes often result in some localized tissue damage around the removed portions

Fractional Photothermolysis (FP) (sometimes referred to as fractional resurfacing) is a laser-assisted treatment that produces a pattern of microscopic treatment zones (MTZs) in biological tissue. The concept of fractional thermolysis is described, e.g., in D. Manstein et al., Fractional photothermolysis: a new concept for cutaneous remodeling using microscopic patterns of thermal injury, Lasers in surgery and medicine 34, 426-438 (2004). FP can be performed in either non-ablative (nFP) or ablative (aFP) modalities. nFP generates MTZs that are small zones of thermally damaged (heated) tissue, whereas aFP generates MTZs that are characterized by a central “hole” of physically-removed (ablated or vaporized) tissue, typically surrounded by a cuff or layer of thermally damaged tissue. The width or diameter of the MTZs are typically less than 1 mm, and often less than about 0.5 mm. Fractional photothermolysis techniques are characterized by direct exposure of only a small fraction of the tissue to the laser radiation (typically an areal fraction of about 5-30%), with most of the tissue being spared or unexposed. Fractional photothermolysis (ablative or non-ablative) is currently used for a wide spectrum of dermatological indications including, but not limited to, treatment of dyschromia, rhytides, photodamaged skin, and various kind of scars including acne, surgical and burn scars.

Photodynamic therapy (PDT) has been used successfully for local cancer therapy. Various types of cancer have been treated with PDT including, but not limited to, skin cancer, lung cancer, bile duct cancer, and pancreatic cancer. The response to PDT treatment is dependent on the cancer type and cell lines present. For example, PDT of intradermally inoculated CT26 wild-type (CT26WT) colon cancer cells was observed to induce only local tumor regression followed by recurrence, as described, e.g., by P. Mroz et al., Photodynamic therapy of tumors can lead to development of systemic antigen-specific immune response, PloS one, 5(12):e15194 (2010). CT26WT is a clone of the N-nitoroso-N-methylurethan (NMU)-induced undifferentiated colon carcinoma. PDT of an intradermally inoculated CT26.CL25 tumor was also observed to induce local remission as well as a systemic tumor-specific immune response, resulting in regression of a remote, untreated antigen-positive tumor.

The CT26.CL25 tumor cell is a clone generated by transduction with lacZ gene encoding beta-galactosidase (beta-gal) antigen to CT26WT. It has thus been observed that PDT is able to induce a systemic, tumor specific anti-tumor immunity. However, PDT has some shortcomings because it is a drug-device combination treatment that requires the administration of the photosensitizing drug in a dose dependent and time-sensitive manner. The PDT effect also depends on the bioavailability of the photosensitizer and requires an oxygen rich environment. Both requirements can be a challenge within tumors, which are often characterized by blood vessel compression and hypoxemia due to the tumor growth. As most non-dermatological tumors require systemic application of the photosensitizer, the resulting requirement for prolonged light avoidance of patients is another downside of systemically delivered PDT.

Ablative FP has been used previously in combination with photodynamic therapy (PDT) to treat skin cancer; however, in conjunction with this indication, FP is mainly used to provide enhanced topical delivery of the photosensitizing drug. Non-ablative FP has been used to treat precancerous skin lesions (actinic keratoses). However such treatments have been limited to direct irradiation of local skin regions, and no studies to date have investigated production of systemic effects using FP methods.

Ablative energy has also been used to treat tumors directly by ablating/removing the entire tumor (often with a small degree of surrounding healthy tissue) using an ablative laser energy source. While extensive and homogenous irradiation of tumors may be desirable for tumor destruction, such “full-irradiation” approaches have potential downsides. For example, the substantially complete destruction of the tumor tissue also destroys nearby immune competent cells that might be helpful to trigger an immune response. This is of particular concern, e.g., in radiation therapy because immune competent cells have a low damage threshold and might be even more vulnerable to a full-irradiation treatment than the tumor cells themselves. Conventional ablative treatments are designed to destroy the tumor, but not to necessarily trigger an immune response. The death pathway varies with different thermal doses, and it is not clear which pathway, if any, might be most effective for stimulating an immune response.

Accordingly, it is desirable to provide new cancer treatments that may be well-tolerated by the body and produce desirable effects such as an enhanced local and/or systemic anti-tumor immune responses, and improved efficacy of existing treatments.

### SUMMARY

Embodiments of the present disclosure can be used to produce a local immune response in cancer tissue and/or enhance effectiveness of cancer treatment in a subject through application of an ablative fractional laser procedure, a checkpoint inhibitor, a TLR7 agonist, or combinations thereof. In certain embodiments, the fractional laser procedure induces a localized immune response in the tumor or lesion. In such embodiments, ablation or removal of tissue from the tumor or lesion is not necessary or required.

Accordingly, one aspect provided herein relates to a method for treating cancer in a subject, the method comprising: (a) administering at least one drug to a subject having a tumor, and (b) contacting tissue of the tumor with a fractional laser, thereby treating cancer in the subject.

In one embodiment of this aspect and all other aspects provided herein, the at least one drug is administered systemically.

In another embodiment of this aspect and all other aspects provided herein, the at least one drug is an immune checkpoint inhibitor.

In another embodiment of this aspect and all other aspects provided herein, the immune checkpoint inhibitor is an inhibitor of PD1, PDL1, TIM-3, or CTLA4.

In another embodiment of this aspect and all other aspects provided herein, the immune checkpoint inhibitor is ipilimumab, tremelimumab, nivolumab, or pembrolizumab.

In another embodiment of this aspect and all other aspects provided herein, the at least one drug is administered locally.

In another embodiment of this aspect and all other aspects provided herein, the at least one drug is administered topically or injected into the tumor tissue.

In another embodiment of this aspect and all other aspects provided herein, the at least one drug is an agonist of TLR3, TLR7, TLR8 or TLR9.

In another embodiment of this aspect and all other aspects provided herein, the TLR7 agonist is imiquimod, reiquimod, or gardiquimod.

In another embodiment of this aspect and all other aspects provided herein, the method further comprises administering at least two drugs.

In another embodiment of this aspect and all other aspects provided herein, the at least two drugs comprise imiquimod and at least one immune checkpoint inhibitor.

In another embodiment of this aspect and all other aspects provided herein, the step of administering a drug to the subject is performed at least twice.

In another embodiment of this aspect and all other aspects provided herein, the step of contacting tumor tissue with the fractional laser is performed at least twice.

In another embodiment of this aspect and all other aspects provided herein, the administering step and the contacting step are performed simultaneously.

In another embodiment of this aspect and all other aspects provided herein, the administering step is performed before or after the contacting step.

In another embodiment of this aspect and all other aspects provided herein, the cancer is melanoma. In another embodiment of this aspect and all other aspects provided herein, the cancer is pancreatic cancer.

In another embodiment of this aspect and all other aspects provided herein, the fractional laser is a CO₂ laser.

In another embodiment of this aspect and all other aspects provided herein, the fractional laser penetrates to a depth of at least 0.1 mm (e.g., at least 0.2 mm, at least 0.3 mm, at least 0.4 mm, at least 0.5 mm, at least 1 mm, at least 1.5 mm, at least 2 mm, at least 2.5 mm, at least 3 mm, at least 3.5 mm, at least 4 mm, at least 4.5 mm, at least 5 mm etc.) into the tumor tissue.

In another embodiment of this aspect and all other aspects provided herein, treatment with the fractional laser induces a local immune response in the tumor tissue.

In another embodiment of this aspect and all other aspects provided herein, treatment with the fractional laser does not damage the stratum corneum.

In another embodiment of this aspect and all other aspects provided herein, treatment with the fractional laser does not induce scarring or crusting of the tumor tissue.

In another embodiment of this aspect and all other aspects provided herein, the area of treatment comprises at least 0.25 mm². In another embodiment of this aspect and all other aspects provided herein, the area of treatment comprises at least 0.25 mm² and up to the entire surface of the lesion. In other embodiments of this aspect and all other aspects described herein, the area of treatment comprises at least 5% of the tumor or lesion area; in other embodiments the area of treatment comprises at least 10%, at least 15%, at least 20%, at least 25%, at least 30%, at least 40%, at least 50%, at least 60%, at least 70% at least 75%, at least 80%, at least 85%, at least 90%, at least 95%, at least 99%, or more of the tumor or lesion area.

In another embodiment of this aspect and all other aspects provided herein, the volume of treatment (e.g., within or near a tumor) comprises at least 5 mm³, at least 10 mm³, at least 15 mm³, at least 20 mm³, at least 25 mm³, at least 30 mm³, at least 35 mm³, at least 40 mm³, at least 45 mm³, at least 50 mm³, at least 55 mm³, at least 60 mm³, at least 65 mm³, at least 70 mm³, at least 75 mm³, at least 80 mm³, at least 85 mm³, at least 90 mm³, at least 95 mm³, at least 100 mm³, or more.

In another embodiment of this aspect and all other aspects provided herein, the energy of the fractional laser is 1 mJ to 200 mJ. In another embodiment of this aspect and all other aspects described herein, the energy of the fractional laser is in the range of 1 mJ to 5 mJ, 1 mJ to 10 mJ, 1 mJ to 20 mJ, 1 mJ to 30 mJ, 1 mJ to 40 mJ, 1 mJ to 50 mJ, 1 mJ to 75 mJ, 1 mJ to 100 mJ, 1 mJ to 125 mJ, 1 mJ to 150 mJ, 1 mJ to 175 mJ, 25 mJ to 200 mJ, 50 mJ to 200 mJ, 50 mJ-100 mJ, 75 mJ-100 mJ, 75-125 mJ, 80-110 mJ, 100 mJ to 200 mJ, 125 mJ-200 mJ, 150 mJ to 200 mJ, 175 mJ to 200 mJ, 50 mJ to 100 mJ, 25 mJ to 75 mJ, 25 mJ to 150 mJ, or any range therebetween.

In another embodiment of this aspect and all other aspects provided herein, approximately 50 mJ-110 mJ (e.g., 100 mJ) of energy is used for a superficial lesion and approximately 200 mJ of energy is used for a deep tumor.

In another embodiment of this aspect and all other aspects provided herein, the pulse duration of the fractional laser is 100 usec to 10 msec.

In another embodiment of this aspect and all other aspects provided herein, the pulse duration of the fractional laser is 2 msec. In other embodiments of this aspect and all other aspects provided herein, the pulse duration of the fractional laser is between 100 usec to 5 msec, 100 usec to 1 msec, 100 usec to 500 usec, 100 usec to 250 usec, 100 usec to 200 usec, from 250 usec to 10 msec, from 500 usec to 10 msec, from 750 usec to 10 msec, from 1 msec to 10 msec, from 2 msec to 10 msec, from 5 msec to 10 msec, from 1 msec to 5 msec, from 1 msec to 3 msec or any range therebetween.

In another embodiment of this aspect and all other aspects provided herein, the spot size of the fractional laser is 10 um to 1 mm. In other embodiments of this aspect and all other aspects provided herein, the spot size of the fractional laser is in the range of 10 um to 750 um, 10 um to 500 um, 10 um to 250 um, 10 um to 150 um, 10 um to 100 um, 10 um to 50 um, 10 um to 25 um, 400 um to 1 mm, 500 um to 1 mm, 600 um to 1 mm, 700 um to 1 mm, 800 um to 1 mm, 900 um to 1 mm, 50 um to 750 um, 75 um to 500 um, 100 um to 500 um, 250 um to 500 um, or any range therebetween.

In another embodiment of this aspect and all other aspects provided herein, the penetration depth is ⅓ the depth of the tumor. In other embodiments the penetration depth is at least 40% of the depth of the tumor, at least 50%, at least 60%, at least 70%, at least 75%, at least 80%, at least 90%, at least 95%, at least 99% the depth of the tumor. In some embodiments, the penetration depth does not need to penetrate the tumor tissue itself, provided that the fractional laser treatment induces a localized immune response within the tumor or along the borders of the tumor.

In another embodiment of this aspect and all other aspects provided herein, the fractional laser reaches at least 0.5% of the tumor volume, e.g., at least 1%, at least 1.5%, at least 2%, at least 2.25%, at least 2.5%, at least 3%, at least 3.5%, at least 4%, at least 5%, at least 10% of the tumor volume. In another embodiment of this aspect and all other aspects provided herein, the fractional laser reaches less than 0.5% of the tumor volume, e.g., less than 1%, less than 1.5%, less than 2%, at less than 2.25%, less than 2.5%, less than 3%, less than 3.5%, less than 4%, less than 5%, or less than 10% of the tumor volume.

Another aspect described herein relates to a method of promoting resistance of a subject to recurrence of a cancer, the method comprising: (a) administering at least one drug to a subject having a tumor, and (b) contacting tissue of the tumor with a fractional laser, thereby promoting resistance of the subject to a recurrence of the cancer.

In one embodiment of this aspect and all other aspects described herein, the at least one drug is administered systemically.

In another embodiment of this aspect and all other aspects described herein, the at least one drug is an immune checkpoint inhibitor.

In another embodiment of this aspect and all other aspects described herein, the immune checkpoint inhibitor is an inhibitor of PD1, PDL1, TIM-3, or CTLA4.

In another embodiment of this aspect and all other aspects described herein, the immune checkpoint inhibitor is ipilimumab, tremelimumab, nivolumab, or pembrolizumab.

In another embodiment of this aspect and all other aspects described herein, the at least one drug is administered locally.

In another embodiment of this aspect and all other aspects described herein, the at least one drug is administered topically or injected into the tumor tissue.

In another embodiment of this aspect and all other aspects described herein, the at least one drug is an agonist of TLR3, TLR7, TLR8 or TLR9.

In another embodiment of this aspect and all other aspects described herein, the TLR7 agonist is imiquimod, reiquimod, or gardiquimod.

In another embodiment of this aspect and all other aspects described herein, the method further comprises administering at least two drugs.

In another embodiment of this aspect and all other aspects described herein, the at least two drugs comprise imiquimod and at least one immune checkpoint inhibitor.

In another embodiment of this aspect and all other aspects described herein, the step of administering a drug to the subject is performed at least twice.

In another embodiment of this aspect and all other aspects described herein, the step of contacting tumor tissue with the fractional laser is performed at least twice.

In another embodiment of this aspect and all other aspects described herein, the administering step and the contacting step are performed simultaneously.

In another embodiment of this aspect and all other aspects described herein, the administering step is performed before or after the contacting step.

In another embodiment of this aspect and all other aspects described herein, the cancer is melanoma or metastatic melanoma.

In another embodiment of this aspect and all other aspects described herein, the fractional laser is a CO₂ laser.

In another embodiment of this aspect and all other aspects described herein, the fractional laser penetrates to a depth of at least 0.1 mm into the tumor tissue.

In another embodiment of this aspect and all other aspects described herein, treatment with the fractional laser induces a local immune response in the tumor tissue.

In another embodiment of this aspect and all other aspects described herein, treatment with the fractional laser does not damage the stratum corneum.

In another embodiment of this aspect and all other aspects described herein, treatment with the fractional laser does not induce scarring or crusting of the tumor tissue.

In another embodiment of this aspect and all other aspects described herein, the area of treatment comprises at least 0.25 mm².

In another embodiment of this aspect and all other aspects described herein, the energy of the fractional laser is 1 mJ to 200 mJ.

In another embodiment of this aspect and all other aspects described herein, 50 mJ of energy is used for a superficial lesion and 200 mJ of energy is used for a deep tumor.

In another embodiment of this aspect and all other aspects described herein, the energy of the fractional laser is 100 mJ.

In another embodiment of this aspect and all other aspects described herein, the pulse duration of the fractional laser is 100 usec to 10 msec.

In another embodiment of this aspect and all other aspects described herein, the pulse duration of the fractional laser is 2 msec.

In another embodiment of this aspect and all other aspects described herein, the spot size of the fractional laser is 10 um to 1 mm.

In another embodiment of this aspect and all other aspects described herein, the penetration depth of the fractional laser is ⅓ the depth of the tumor.

### BRIEF DESCRIPTION OF THE FIGURES

FIGS. 1A-1C. UVB-associated mutations enhance anti-tumor immunity and response to PD-1 blockade in a syngeneic implantable melanoma model. FIG. 1A, Overview of genetic alterations in UVB-mutagenized clones UV2 and UV3 relative to their parental melanoma cell line. Types of base substitutions and classes of single nucleotide variants (SNVs) are shown. FIG. 1B, Parental, UV2, and UV3 melanoma growth in NSG mice and corresponding survival. Data are shown as mean tumor size ±SD (n=5 per group). n.s. not significant (two tailed t-test and log-rank test). FIG. 1C, Parental, UV2, and UV3 melanoma growth in C57BL/6 mice and corresponding survival. Mice received anti PD-1 or isotype-matched control antibody on days 8, 10, 12, 14, and 16 after tumor cell inoculation. Mean UV tumor sizes did not differ significantly from parental melanoma sizes on day 8. Data are shown as mean tumor size ±SD (n=5 per group). For survival analysis, *p<0.05 comparing UV2 anti-PD-1 to parental anti-PD-1; **p<0.01, comparing UV clone isotype to parental isotype, or UV3 anti-PD-1 to parental anti-PD-1 (log-rank test).

FIGS. 2A-2E. Introduction of putative neoantigens promotes recruitment of tumor infiltrating immune cells and is associated with T cell dysfunction that is reversed by PD-1 blockade. FIG. 2A, GSEA of RNA-sequencing data from bulk tumor grafts in C57BL/6 hosts. Representative top-scoring KEGG gene sets enriched in UV2 compared to parental melanomas with nominal p values<0.01 are shown. FDR, false discovery rate. FIG. 2B, CD3 expression in parental and UV2 melanomas harvested 5 days after initiation of therapy was assayed via immunohistochemistry in 9 randomly selected intratumoral ×20 fields from 3 different mice per group (representative fields shown). Data are shown as mean±SEM. ****p<0.0001; n.s. not significant (Tukey's multiple comparisons test). FIGS. 2C & 2D Immune infiltrates in tumors (TILs) and draining lymph nodes (dLNs) harvested 5 days after anti-PD-1 therapy initiation, characterized by flow cytometry. Numbers of CD8+ and Treg T cells (FIG. 2C) and ratios of CD8+ T cells to Tregs (CD4+FoxP3+) (FIG. 2D) are shown, as are the proportions of CD8+ T cells positive for Ki67 or granzyme B (FIG. 2D). Data are shown as mean±SEM (n=12 pooled to 6 per group). *p<0.05; **p<0.01; ***p<0.001; ****p<0.0001 (two tailed t-test). FIG. 2E, TCR sequencing of bulk melanomas from C57BL/6 hosts treated with isotype-matched control antibody. Richness (unique complementarity-determining region 3 [CDR3] rearrangements), entropy (diversity of rearrangements), and clonality are shown for parental (n=6) and UV2 (n=4). Data are shown as mean±SD. n.s, not significant (two-tailed t-test).

FIGS. 3A-3C. Addition of imiquimod and aFP improves response of poorly immunogenic melanoma and PDAC to checkpoint blockade and confers long term immunity. FIG. 3A, Survival of C57BL/6 mice following parental melanoma inoculation (day 0) and combination treatments using anti-PD-1, aFP, and imiquimod administered on the days indicated (n=10 mice per group). ***p<0.001 comparing triple therapy to anti-PD-1 (log rank test). Spider plots show growth of individual tumors in C57BL/6 mice on treated (left) and untreated (right) flanks after therapy with aFP and/or imiquimod administered to the treated tumor only. Pie charts show percent complete responses. FIG. 3B, Survival of C57BL/6 mice following parental melanoma inoculation and treatment with isotype-matched antibodies (n=9), anti-PD-1+anti-CTLA-4 (n=16), or quadruple therapy using PD-1 and CTLA-4 blockade, aFP, and imiquimod (n=17). *p<0.05 comparing quadruple therapy to anti-PD-1+anti-CTLA-4 (log-rank test). FIG. 3C, Triple therapy induces tumor regression in a mouse model of poorly immunogenic PDAC. Survival of C57BL/6 mice following subcutaneous inoculation of KPC pancreatic cancer cells and treatment with isotype-matched antibody, anti-PD-1, or triple therapy using anti-PD-1, aFP, and imiquimod (n=5 per group). **p<0.01 comparing triple therapy to anti-PD-1 (log-rank test).

FIGS. 4A-4H. Imiquimod and aFP synergize with immune checkpoint blockade to enhance the number and function of tumor-infiltrating T cells and induce responses against wildtype tumor-lineage antigens. FIG. 4A, Representative top-scoring KEGG gene sets enriched in bulk parental melanomas in C57BL/6 mice treated with triple therapy (anti-PD-1+aFP+imiquimod) compared to anti-PD-1 monotherapy with nominal p values<0.01. FDR, false discovery rate. FIG. 4B, CD3 expression in parental melanomas harvested 5 days after initiation of therapy was assayed via immunofluorescence in 6 randomly selected intratumoral ×20 fields (representative fields shown). Data are shown as mean±SEM. *p<0.05; ****p<0.0001; n.s. not significant (Tukey's multiple comparisons test). FIG. 4C, Immune infiltrates in contralateral (untreated flank) tumors and draining lymph nodes harvested 5 days after initiation of i.p. antibody treatments, and application of aFP and imiquimod to treated flank tumors, characterized by flow cytometry. Ratios of CD8+ T cells to Tregs (CD4+FoxP3+) and proportion of CD8+ T cells that are positive for granzyme B in tumors are shown, as well as proportion of PD-L2+ CD11c+ dendritic cells in draining lymph nodes. Top panel: n=7 for isotype control, aFP, imiquimod, and aFP+imiquimod, n=9 for anti-PD-1; asterisks indicate significance compared with control by Dunnett's multiple comparisons test. Bottom panel: n=12 pooled to 6 per group; asterisks indicate significance compared with anti-PD-1 (for double or triple combinations) or compared with anti-PD-1+anti-CTLA-4 (for quadruple combination) by Dunnett's multiple comparisons test. Data are shown as mean±SEM. *p<0.05; **p<0.01; ***p<0.001;***p<0.0001. FIG. 4D, anti-CD8 or isotype-matched control antibodies were administered every 3 days, beginning 1 week before inoculation of parental melanoma cells into C57BL/6 mice. All mice received triple therapy with imiquimod, FP, and anti-PD-1. n=10 mice per group. ****p<0.0001 (log-rank test). FIG. 4E, TCR sequencing of bulk melanomas from C57BL/6 hosts treated with isotype-matched control antibody, anti-PD-1, or triple therapy (imiquimod+aFP+anti-PD-1). Richness (unique CDR3 rearrangements), entropy (diversity of rearrangements), and clonality are shown for parental (n=6) and UV2 (n=4). Data are shown as mean±SD. n.s, not significant (two-tailed t-test). FIG. 4F, GSEA plots showing enrichment of pigmentation gene set GO:0043473 in ipilimumab responders in the low neoantigen load subset of patients as well as in triple therapy-treated mouse parental melanomas. ES, enrichment score. FIG. 4G, CD8+ T cells from treated flank parental melanomas (TILs) and dLNs 5 days after initiation of therapy were evaluated for binding to gp100:H-2Db tetramer (n=8 mice per group). Data are shown as mean±SEM. ***p<0.001; n.s. not significant (Tukey's multiple comparisons test). FIG. 4H, At left, survival of parental (n=3) or UV2 (n=3) melanoma-bearing mice with complete responses to triple therapy, following rechallenge with parental melanoma cells. At right, survival of parental melanoma-bearing mice with complete responses to triple therapy (n=8), anti-PD-1+aFP (n=2), or anti-PD-1+imiquimod (n=3), following challenge with B16-F10 melanoma cell inoculation. **p<0.01; ***p<0.001 (log-rank test).

FIGS. 5A-5C. Characterization of UV2 and UV3 melanoma cell lines. FIG. 5A, Growth rates of parental melanoma cells and UV clones were monitored after rescue from 16 h serum starvation using the Cell-Titer-Glo ATP-based luminescence assay. Data are shown as mean±SD (technical triplicates) and are representative of 2 independent experiments. FIG. 5B, Similar growth rates of parental, UV2, and UV3 melanoma cells after rescue from 16 h serum starvation as measured by cell counting. Data are shown as mean±SD (technical triplicates) and are representative of 2 independent experiments. n.s. not significant (two-tailed t-test). FIG. 5C, Representative flow plots for PD-1, PD-L1, and MHC class I and II expression on mouse melanoma cells with or without IFN-γ stimulation.

FIGS. 6A-6B. RNA-sequencing reveals enhanced cytotoxic activity and upregulation of T cell dysfunction markers in UV2 melanomas compared to matched parental melanomas. FIG. 6A, Cytolytic activity defined as the log-average (geometric mean) of granzyme A and perform 1 RNA expression per million transcripts in bulk mouse tumors harvested 5 days after initiation of anti-PD-1 or isotype-matched antibody administration. Data are shown as mean±SD (n=3 per group). *p<0.05 (two-tailed t-test). FIG. 6B, mRNA expression of inhibitory and exhaustion markers that differed significantly between UV2 and parental bulk melanomas. Floating bars show minimum and maximum values with a line at the mean (n=3 per group). *p<0.05; **p<0.01; ***p<0.001 as determined by DESeq2 analysis.

FIGS. 7A-7F. Imiquimod and aFP synergize with anti-PD-1, anti-CTLA-4, and dual anti-PD-1+anti-CTLA-4 and induce an abscopal effect. FIG. 7A, TCGA patients with melanomas in the top quartile for TLR7 expression had significantly longer survival than patients with melanomas in the bottom quartile for TLR7 expression. FIG. 7B, Tumor growth of parental melanomas following combination therapy. Data from FIG. 3A are presented as mean volumes of both treated and untreated flank tumors ±SEM (n=10 mice per group). Corresponding survival data are shown in FIGS. 3A & 3C. FIG. 7C, Tumor growth and survival of C57BL/6 mice following parental melanoma inoculation and combination treatments using anti-CTLA-4, aFP, and imiquimod according to the indicated schedule (n=8 per group). Data are shown as mean volumes of tumors on both flanks ±SEM. **p<0.001 comparing triple therapy to anti-CTLA-4 (log-rank test). FIG. 7D, Comparison of tumor growth on the left versus right flanks of C57BL/6 mice after triple therapy with aFP and imiquimod, administered to the left tumors only, plus systemic anti-CTLA-4 (n=8 mice per group) or anti-PD-1 (n=10 mice per group). Data are shown as mean tumor size ±SEM. n.s., not significant (two tailed t-test comparison of left versus right tumors). FIG. 7E, Parental melanoma growth in C57BL/6 mice following isotype control (n=9), anti-PD-1+anti-CTLA-4 (n=16), or quadruple therapy using PD-1 and CTLA-4 blockade, aFP, and imiquimod (n=17). Data are shown as mean volumes of tumors on both flanks ±SEM. Corresponding survival data are shown in FIG. 3B. FIG. 7F, KPC pancreatic ductal adenocarcinoma growth following inoculation into C57BL/6 mice and treatment with isotype-matched control, anti-PD-1, or triple therapy using anti-PD-1, aFP, and imiquimod (n=5 per group). Data are shown as mean volumes of tumors on both flanks ±SEM. Corresponding survival data are shown in FIG. 3C.

FIGS. 8A-8C. Combination immunotherapy improves T cell responses and is associated with markers of increased dendritic cell infiltration and function. FIG. 8A, Immune infiltrates in untreated and treated flank tumors (TILs) and draining lymph nodes (dLNs) harvested 5 days after therapy initiation characterized by flow cytometry. Ratios of CD8+ T cells to Tregs (CD4+FoxP3+) and proportion of CD8+ T cells that are positive for granzyme B in tumors are shown, as well as proportion of PD-L2+ CD11c+ dendritic cells in draining lymph nodes. Top panel: n=7 for isotype control, aFP, imiquimod, and aFP+imiquimod; n=9 for anti-PD-1; asterisks indicate significance compared with control by Dunnett's multiple comparisons test. Bottom panel: n=12 pooled to 6 per group, asterisks indicate significance compared with anti-PD-1 (for double or triple combinations) or compared with anti-PD-1+anti-CTLA-4 (for quadruple combination) by Dunnett's multiple comparisons test. Untreated flank tumor data are the same as shown in FIG. 4C. Data are shown as mean±SEM. FIG. 8B, Overall survival (OS) and predicted neoantigen numbers of 40 patients with whole-exome and RNA sequencing data available from pre-treatment melanoma biopsies as reported in Van Allen et al 2015. The low neoantigen subset was defined as patients with fewer than 100 predicted neoantigens with <50 nM binding affinities for HLA class I. Ipilimumab responders and non-responders are shown. FIG. 8C, Survival of mice with complete responses against parental melanomas following triple therapy (n=3) or quadruple therapy (n=3) after challenge with KPC cell inoculation. n.s., no significant difference between parental survivors and naïve C57BL/6 mice (log-rank test).

### DETAILED DESCRIPTION

Provided herein are methods that can be used to produce a local immune response in cancer tissue and/or enhance effectiveness of cancer treatment in a subject through application of one or more combinations of: an ablative fractional laser procedure, a checkpoint inhibitor, and an endosomal TLR agonist (e.g., agonist of TLR3, TLR7, TLR8 or TLR9).

### Definitions

As used herein, the terms “fractional treatment,” “fractional laser treatment,” and “fractional photothermolysis” can generally describe the generation of damage, heating, and/or ablation/vaporization of multiple small individual exposure areas of tissue (e.g., generally having at least one dimension that is less than about 1 mm) of biological tissue or other tissue. Such damage can be produced by mechanical means or by exposing the tissue to energy, such as directed optical energy produced by a laser. After fractional treatment, substantially undamaged, unablated, and/or unheated areas or volumes of tissue are present between the irradiated, damaged, and/or ablated/vaporized regions. The individual exposure areas can be, for example, oval, circular, arced and/or linear in shape.

The terms “nonablative” and “subablative” as used herein can refer to processes that do not involve vaporization or other energy-based removal of biological tissue or other material from the site of treatment at the time of treatment.

As used herein, the term “immune checkpoint inhibitor” can refer to molecules that may totally or partially reduce, inhibit, interfere with or modulate one or more checkpoint proteins, which in turn regulate T-cell activation or function. Numerous checkpoint proteins are known, such as CTLA-4 and its ligands CD 80 and CD86; PD1 with its ligands PDL1 and PDL2 (Pardoll, Nature Reviews Cancer 12: 252-264, 2012), and TIM3. These proteins are responsible for co-stimulatory or inhibitory interactions of T-cell responses. Immune checkpoint proteins regulate and maintain self-tolerance and the duration and amplitude of physiological immune responses. Immune checkpoint inhibitors include antibodies that bind a checkpoint protein or constructs employing the antigen-binding domain of an antibody.

The terms “decrease”, “reduced”, “reduction”, or “inhibit” are all used herein to mean a decrease or lessening of a property, level, or other parameter by a statistically significant amount. In some embodiments, “reduce,” “reduction” or “decrease” or “inhibit” typically means a decrease by at least 10% as compared to a reference level (e.g., the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase” or “enhance” or “activate” are all used herein to generally mean an increase of a property, level, or other parameter by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, at least about a 20-fold increase, at least about a 50-fold increase, at least about a 100-fold increase, at least about a 1000-fold increase or more as compared to a reference level.

The term “pharmaceutically acceptable” can refer to compounds and compositions which can be administered to a subject (e.g., a mammal or a human) without undue toxicity.

As used herein, the term “pharmaceutically acceptable carrier” can include any material or substance that, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. The term “pharmaceutically acceptable carriers” excludes tissue culture media.

As used herein, the term “comprising” means that other elements can also be present in addition to the defined elements presented. The use of “comprising” indicates inclusion rather than limitation.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Further, unless otherwise required by context, singular terms shall include pluralities and plural terms shall include the singular.

It should be understood that this invention is not limited to the particular methodologies, protocols, and reagents, etc., described herein and as such can vary therefrom. The terminology used herein is for the purpose of describing particular embodiments only, and is not intended to limit the scope of the present invention, which is defined solely by the claims.

### Fractional Laser Treatment

Embodiments of the present disclosure can provide fractional damage of tumors in combination with one or more further therapies. Such fractional damage can facilitate a local and/or systemic immune response, and/or promote an immune system attack on the tumor. In certain embodiments, such fractional damage to tumor tissue can also enhance the efficacy of other therapies that can be used in combination. Thus in some embodiments, the dose of one or more therapies administered in combination with fractional laser treatment is lower than the dose of the one or more therapies in the absence of fractional laser treatment (e.g., conventional anti-cancer treatment). While well-suited to treatment of skin tumors, including but not limited to melanoma, fractional treatments can also be applied to tumors located elsewhere in the body (e.g., pancreatic cancer).

In certain embodiments, the fractional damage can be generated using an ablative fractional photothermolysis (aFP) procedure. Unlike conventional ablative treatments of tumors, which are directed to complete destruction of the tumor tissue using a laser or other optical energy source, fractional laser radiation treatments involve the generation of a large number of small, discrete treatment zones within a region of the tumor tissue. Accordingly, a region or volume of tissue (e.g., tumor tissue) treated during an aFP procedure, will exhibit a number of discrete microscopic treatment zones (MTZs) where the tissue has been altered (e.g., partially or fully ablated or vaporized) by the laser radiation. These MTZs will be present within a larger volume of tissue that remains substantially unaltered by the laser radiation.

In further embodiments, the MTZs can be formed using other modalities, such as non-laser optical energy, focused ultrasound, radiofrequency (RF) energy, etc. For example, RF energy can be used to form a plurality of MTZs in tissue using a plurality of surface or penetrating (e.g., needle-like) electrodes provided on the tissue surface and/or within the tissue.

When treating skin with fractional laser treatment methods described herein (e.g., for treatment of melanoma), a wide range of treatment effects within the skin can be achieved by varying the laser treatment parameters. These laser treatment parameters can include, for example, wavelength, local irradiance, local fluence, pulse energy, pulse duration, treatment zone size or spot size, treatment zone density, beam diameter, and combinations thereof. Substantially the same parameters can be varied when the area treated is not the skin. Laser energy can be applied internally, e.g., via catheter or during surgery.

For example, the number and density of MTZs can be predetermined by selecting the fractional treatment parameters. In certain embodiments, the fractional treatment can be performed by directing a beam of energy onto a plurality of locations on the surface of the tissue (e.g., tumor tissue) being treated. In further embodiments, a plurality of beams can be directed simultaneously onto a plurality of locations on the tissue surface. The plurality of beams can be provided by a plurality of lasers or laser diodes, or alternatively by splitting a single beam of energy into a plurality of beams using an optical arrangement.

Fractional treatment of tumor tissue can provide an areal fraction of tissue surface that is irradiated that is between about 0.05 and about 0.50 mm². In certain embodiments, the areal fraction can be between about 0.05 and 0.20 mm². Such smaller fractions of treated tissue can better avoid overall bulk heating of the tumor tissue while generating local damage therein. For a particular beam diameter, this areal fraction can be determined as the area of an individual beam cross-section multiplied by the number of distinct beam irradiation locations on a treated surface region, divided by the area of the treated surface region. Similar calculations of areal coverage can be determined, e.g., for different beam shapes and irradiation geometries including, e.g., irradiation patterns that include ellipses, thin lines, etc. by dividing the total area of irradiating energy beams directed onto the treated region divided by the area of the treated region.

In another embodiment of this aspect and all other aspects provided herein, the fractional laser reaches at least 0.5% of the tumor volume, e.g., at least 1%, at least 1.5%, at least 2%, at least 2.25%, at least 2.5%, at least 3%, at least 3.5%, at least 4%, at least 5%, at least 10% of the tumor volume. In another embodiment of this aspect and all other aspects provided herein, the fractional laser reaches less than 0.5% of the tumor volume, e.g., less than 1%, less than 1.5%, less than 2%, at less than 2.25%, less than 2.5%, less than 3%, less than 3.5%, less than 4%, less than 5%, or less than 10% of the tumor volume.

The individual energy beams (which may be pulsed) that are used to create the MTZs in tissue can be generally less than 1 mm in width or diameter. Such width approximately corresponds to the width of the MTZs formed by the beams, and can be well-tolerated by the surrounding tissue and can prevent excessive or widespread disruption of the tumor tissue that could lead to spreading of tumor cells within the patient. In further embodiments, the width of these beams can be less than 0.5 mm, or less than 0.2 mm. Such smaller beam widths can generate MTZs that are narrow enough to disrupt tumor tissue while further reducing the likelihood of unwanted spreading or ‘release’ of tumor cells within the patient. The MTZs can be formed as ablated holes within the tissue, which may partially or completely collapse soon after formation.

The depth of the ablated holes and/or of the MTZs formed during fractional treatment of tumor tissue can be determined using known techniques based on, e.g., the wavelength(s) of energy used, the fluence, cross-sectional area and power of the energy beams, the characteristics of the treated tissue, etc. In general, it is preferable that the MTZs extend to one or more particular depths within the tumor tissue. For example, in certain embodiments, the MTZs can extend to a depth that is at least about ¼ of the distance between the tumor surface and the center of the tumor. The particular depth(s) of the MTZs can be selected based on the size and type of tumor being treated. For example, the depth of the MTZs can be selected such that they extend through an outer layer of the tumor and at least into an interior (or core) region of the tumor. In still further embodiments, characteristics of the fractional treatment can be selected such that the MTZs (e.g., ablated holes) can extend completely through the entire tumor. In some embodiments, the fractional laser penetrates to a depth of at least 0.1 mm (e.g., at least 0.2 mm, at least 0.3 mm, at least 0.4 mm, at least 0.5 mm, at least 1 mm, at least 1.5 mm, at least 2 mm, at least 2.5 mm, at least 3 mm, at least 3.5 mm, at least 4 mm, at least 4.5 mm, at least 5 mm etc.) into the tumor tissue.

In further embodiments, one or more tumors being treated can be located below another exposed tissue surface, such as a skin tissue. The parameters of the fractional treatment can be selected such that the MTZs extend through the overlying tissue, and into or through the tumor as described above. Conventional calculations using known energy and tissue parameters can be performed by one of ordinary skill in the art to provide a set of parameters for the applied energy (e.g., beam width, duration, wavelength, fluence, power, etc.) for specific procedures in accordance with the present disclosure, e.g., to generate MTZs that extend a particular depth into tumor and/or overlying tissue.

In still further embodiments, tumors located within the body (e.g., away from an exposed tissue surface) can also be treated. For such tumors, fractional treatment can be performed by delivering energy to the tumor(s) using a fiberscope, an endoscope, a catheter-disposed arrangement configured to deliver energy, a laparoscopic device, focused ultrasound energy, or the like. In such embodiments, the energy (beam) parameters can be selected to produce MTZs within the tumor tissue as described above.

In certain embodiments, a CO₂ laser can be used to form the MTZs during fractional treatment of tumor tissue. In further embodiments, the energy source can be an erbium laser (e.g., an Er:YAG laser), or another type of laser capable of ablating biological tissue.

In still further embodiments, fractional damage of tumor tissue can be performed non-ablatively, to generate MTZs of intact but thermally-damaged tissue within the tumor. Such non-ablative FP can be performed using an energy source such as, e.g., a pulsed dye laser, a Nd:YAG laser, or an Alexandrite laser. In still further embodiments, MTZs of non-ablative fractional damage can be generated in tumor tissue using focused ultrasound energy having a sufficiently low intensity to avoid ablation of tissue.

In still further embodiments, MTZs can be formed in tumor tissue by generating mechanical damage, e.g., by piercing the tumor tissue with an array of needles or multiple times with a single needle. A diameter of the needles can be less than about 1 mm, e.g., less than 0.5 mm, or about 0.1 to 0.2 mm. In certain embodiments the needle(s) can be heated prior to insertion into the tumor tissue to produce some thermal damage as well as mechanical disruption. For example, the needle(s) can be heated using a heated bath or other hot reservoir, or by providing a controlled amount of radiofrequency (RF) energy to the needle(s).

Because of the small size of the MTZs formed during aFP and other fractional procedures, tissue damage produced in the MTZs is well-tolerated, and can induce a healing response in surrounding healthy tissue. Such effects have been observed in dermatological applications of various types of fractional treatment.

The MTZ sizes (e.g., widths and depths) described herein can facilitate limited exposure of the interior of the tumor to the body's immune system and thereby stimulate or activate an autoimmune response. For example, histology performed following aFP treatments of certain tumor tissues revealed an elevated level of erythrocytes, indicating an enhancement of blood flow within the tumor resulting from the aFP treatment. The apparent increase in blood flow in the tumor can facilitate some limited transport of tumor cells out of the tumor, but can also facilitate access of immune competent cells to the core region of the tumor. For example, the enhanced tissue pressure within the core of rapidly-growing tumors can make the core region inaccessible to immune competent cells, which rely on vascular perfusion of the tumor. Also, despite their observed collapse, and without wishing to be bound by theory, ablated channels (e.g., MTZs) in tumor tissue can facilitate access of immune competent cells to cancer cells within the tumor.

Ablative FP CO₂ laser treatments produce small holes in tissue by vaporization thereof at temperatures exceeding 100° C. This results in a steep temperature gradient surrounding the individual MTZs that include the vaporized holes. This steep temperature gradient exposes tumor cells adjacent to the laser-induced holes to a range of temperatures ranging from the peak temperature down to normal body temperature. Accordingly, without being bound by theory, fractional treatment of tumor tissue using aFP or other energy-based techniques (including, e.g., mechanical damage accompanied by local heating, as can be achieved with insertion of heated needles into tumor tissue) can produce weakened (e.g., thermally-damaged) tumor cells and also facilitate their exposure to components of the body's immune system. Such exposure may facilitate an autoimmune response and/or other responses to the cancerous tissue without ‘overwhelming’ the body's defenses or allowing a large number of active tumor cells to spread through the body after such fractional treatment. In some embodiments, treatment of a tumor with ablative FP is performed using settings that do not cause substantial loss of immune cells in the tumor.

Exposure of cells surrounding the MTZs to a range of temperatures can occur without significant bulk heating in the fractionally-treated tissue volume, indicating a lack of confluent thermal injury within the tumor tissue. This particular thermal injury pattern within the tumor tissue distinguishes aFP treatment of tumor tissue from prior energy-based tumor treatment approaches using physical modalities, such as ionizing radiation therapy or classical thermal ablation approaches, that typically provide a relatively homogenous dose of energy throughout the tumor tissue.

Accordingly, one possible advantage of the thermal damage pattern characteristic of FP treatments is that throughout the tumor, cancer cells are exposed to a range of temperatures that can vary from the normal body temperature of the host up to the vaporization temperatures generated in the MTZs, which may be in excess of 100° C. Although only one specific aFP treatment pattern and pulse energy was utilized in the present study, triggering of a marked systemic immune response was observed despite the minimal amount of overall thermal damage done to the tumor volume. It was estimated that ˜2.4% of the total tumor volume was exposed to the laser and thus thermally damaged.

Also provided herein, in other aspects, are methods for treating cancer in a subject, for example, a method comprising: contacting tissue of a tumor with a fractional laser, thereby treating cancer in the subject. In one embodiment of this aspect and all other aspects provided herein the method for treating cancer does not comprise substantial ablation or removal of tissue from the tumor (i.e., less than 5% of the total tumor tissue is ablated/removed; less than 4%, less than 3.5%, less than 3%, less than 2.5%, less than 2.25%, less than 2%, less than 1.75%, less than 1.5%, less than 1.25%, less than 1%, less that 0.5% or less).

In one embodiment of this aspect and all other aspects provided herein, the fractional laser is a CO₂ laser. In one embodiment of this aspect and all other aspects provided herein, the parameters of the fractional laser are tuned such that the laser is non-ablative.

In another embodiment of this aspect and all other aspects provided herein, the fractional laser penetrates to a depth of at least 0.1 mm (e.g., at least 0.2 mm, at least 0.3 mm, at least 0.4 mm, at least 0.5 mm, at least 1 mm, at least 1.5 mm, at least 1.75 mm, at least 2 mm, at least 2.25 mm, at least 2.5 mm, at least 3 mm, at least 3.5 mm, at least 4 mm, at least 4.5 mm, at least 5 mm, etc.) into the tumor tissue.

In another embodiment of this aspect and all other aspects provided herein, treatment with the fractional laser induces a local immune response in the tumor tissue.

In another embodiment of this aspect and all other aspects provided herein, treatment with the fractional laser does not damage the stratum corneum. In another embodiment of this aspect and all other aspects described herein, the fractional laser treatment does not result in substantial ablation or removal of tissue from the tumor (i.e., less than 5% of the total tumor tissue is ablated/removed).

In another embodiment of this aspect and all other aspects provided herein, treatment with the fractional laser does not induce scarring or crusting of the tumor tissue.

In another embodiment of this aspect and all other aspects provided herein, the area of treatment comprises at least 0.25 mm². In other embodiments of this aspect and all other aspects provided herein, the area of treatment is at least 0.25 mm² up to and including the entire surface of a lesion. In other embodiments of this aspect and all other aspects described herein, the area of treatment comprises at least 5% of the tumor or lesion area; in other embodiments the area of treatment comprises at least 10%, at least 15%, at least 20%, at least 25%, at least 30%, at least 40%, at least 50%, at least 60%, at least 70% at least 75%, at least 80%, at least 85%, at least 90%, at least 95%, at least 99%, or more of the tumor or lesion area.

In another embodiment of this aspect and all other aspects provided herein, the energy of the fractional laser is 1 mJ to 200 mJ. In another embodiment of this aspect and all other aspects described herein, the energy of the fractional laser is in the range of 1 mJ to 5 mJ, lmJ to 10 mJ, 1 mJ to 20 mJ, 1 mJ to 30 mJ, 1 mJ to 40 mJ, 1 mJ to 50 mJ, 1 mJ to 75 mJ, 1 mJ to 100 mJ, 1 mJ to 125 mJ, 1 mJ to 150 mJ, 1 mJ to 175 mJ, 25 mJ to 200 mJ, 50 mJ to 200 mJ, 100 mJ to 200 mJ, 125 mJ-200 mJ, 150 mJ to 200 mJ, 175 mJ to 200 mJ, 50 mJ to 100 mJ, 25 mJ to 75 mJ, 25 mJ to 150 mJ, or any range therebetween. In another embodiment of this aspect and all other aspects provided herein, 40-60 mJ (e.g., 50 mJ) of energy is used for a superficial lesion and 150-200 mJ (e.g., 200 mJ of energy) is used for a deep tumor. In one embodiment, 100 mJ of energy is used for the superficial or deep lesion.

In another embodiment of this aspect and all other aspects provided herein, the pulse duration of the fractional laser is 100 usec to 10 msec. In another embodiment of this aspect and all other aspects provided herein, the pulse duration of the fractional laser is 2 msec. In other embodiments of this aspect and all other aspects provided herein, the pulse duration of the fractional laser is between 100 usec to 5 msec, 100 usec to 1 msec, 100 usec to 500 usec, 100 usec to 250 usec, 100 usec to 200 usec, from 250 usec to 10 msec, from 500 usec to 10 msec, from 750 usec to 10 msec, from 1 msec to 10 msec, from 2 msec to 10 msec, from 5 msec to 10 msec, from 1 msec to 5 msec, from 1 msec to 3 msec or any range therebetween.

In another embodiment of this aspect and all other aspects provided herein, the spot size of the fractional laser is 10 um to 1 mm. In other embodiments of this aspect and all other aspects provided herein, the spot size of the fractional laser is in the range of 10 um to 750 um, 10 um to 500 um, 10 um to 250 um, 10 um to 150 um, 10 um to 100 um, 10 um to 50 um, 10 um to 25 um, 400 um to 1 mm, 500 um to 1 mm, 600 um to 1 mm, 700 um to 1 mm, 800 um to 1 mm, 900 um to 1 mm, 50 um to 750 um, 75 um to 500 um, 100 um to 500 um, 250 um to 500 um, or any range therebetween.

In another embodiment of this aspect and all other aspects provided herein, the penetration depth is at least ⅓ (33%) the depth of the tumor. In other embodiments the penetration depth is at least 40% of the depth of the tumor, at least 50%, at least 60%, at least 70%, at least 75%, at least 80%, at least 90%, at least 95%, at least 99% the depth of the tumor. In some embodiments, the penetration depth does not need to penetrate the tumor tissue itself, provided that the fractional laser treatment induces a localized immune response within the tumor or along the borders of the tumor.

### Immune Checkpoint Inhibitors

The immune system has multiple inhibitory pathways that are critical for maintaining self-tolerance and modulating immune responses. In T-cells, the amplitude and quality of response is initiated through antigen recognition by the T-cell receptor and is regulated by immune checkpoint proteins that balance co-stimulatory and inhibitory signals.

Cytotoxic T-lymphocyte associated antigen 4 (CTLA-4) is an immune checkpoint protein that down-regulates pathways of T-cell activation (Fong et al., Cancer Res. 69(2):609-615, 2009; Weber Cancer Immunol. Immunother, 58:823-830, 2009). Blockade of CTLA-4 has been shown to augment T-cell activation and proliferation. Inhibitors of CTLA-4 include anti-CTLA-4 antibodies. Anti-CTLA-4 antibodies bind to CTLA-4 and block the interaction of CTLA-4 with its ligands CD80/CD86 expressed on antigen presenting cells, thereby blocking the negative down regulation of the immune responses elicited by the interaction of these molecules. Examples of anti-CTLA-4 antibodies are described in U.S. Pat. Nos. 5,811,097; 5,811,097; 5,855,887; 6,051,227; 6,207,157; 6,682,736; 6,984,720; and 7,605,238. One anti-CDLA-4 antibody is tremelimumab, (ticilimumab, CP-675,206). In one embodiment, the anti-CTLA-4 antibody is ipilimumab (also known as 10D1, MDX-D010) a fully human monoclonal IgG antibody that binds to CTLA-4. Ipilimumab is marketed under the name Yervoy™ and has been approved for the treatment of unresectable or metastatic melanoma.

Further examples of checkpoint molecules that can be targeted for blocking or inhibition include, but are not limited to, PDL2, B7-H3, B7-H4, BTLA, HVEM, GALS, VISTA, KIR, 2B4 (belongs to the CD2 family of molecules and is expressed on all NK, γδ, and memory CD8+(αβ) T cells), CD160 (also referred to as BY55), CGEN-15049, CHK 1 and CHK2 kinases, A2aR, TIGIT, DD1-α, TIM-3, Lag-3, and various B-7 family ligands. B7 family ligands include, but are not limited to, B7-1, B7-2, B7-DC, B7-H1, B7-H2, B7-H3, B7-H4, B7-H5, B7-H6 and B7-H7.

Another immune checkpoint protein is programmed cell death 1 (PD-1). PD1 limits the activity of T cells in peripheral tissues at the time of an inflammatory response to infection and limits autoimmunity. PD1 blockade in vitro enhances T-cell proliferation and cytokine production in response to a challenge by specific antigen targets or by allogeneic cells in mixed lymphocyte reactions. A strong correlation between PD1 expression and response was shown with blockade of PD1 (Pardoll, Nature Reviews Cancer, 12: 252-264, 2012). PD1 blockade can be accomplished by a variety of mechanisms including antibodies that bind PD1 or its ligand, PDL1. Examples of PD1 and PDLL blockers are described in U.S. Pat. Nos. 7,488,802; 7,943,743; 8,008,449; 8,168,757; 8,217,149, and PCT Published Patent Application Nos: WO03042402, WO2008156712, WO2010089411, WO2010036959, WO2011066342, WO2011159877, WO2011082400, and WO2011161699. In certain embodiments the PD1 blockers include anti-PD-L1 antibodies. In certain other embodiments the PD1 blockers include anti-PD1 antibodies and similar binding proteins such as nivolumab (MDX 1106, BMS 936558, ONO 4538), a fully human IgG4 antibody that binds to and blocks the activation of PD-1 by its ligands PD-L1 and PD-L2; lambrolizumab (MK-3475 or SCH 900475), a humanized monoclonal IgG4 antibody against PD-1; CT-011 a humanized antibody that binds PD1; AMP-224, a fusion protein of B7-DC; an antibody Fc portion; BMS-936559 (MDX-1105-01) for PD-L1 (B7-H1) blockade. Other immune-checkpoint inhibitors include lymphocyte activation gene-3 (LAG-3) inhibitors, such as IMP321, a soluble Ig fusion protein (Brignone et al., 2007, J. Immunol. 179:4202-4211). Other immune-checkpoint inhibitors include B7 inhibitors, such as B7-H3 and B7-H4 inhibitors. In particular, the anti-B7-H3 antibody MGA271 (Loo et al., 2012, Clin. Cancer Res. July 15 (18) 3834). Also included are TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94).

Additional anti-CTLA4 antagonists include, but are not limited to, the following: any inhibitor that is capable of disrupting the ability of CD28 antigen to bind to its cognate ligand, to inhibit the ability of CTLA4 to bind to its cognate ligand, to augment T cell responses via the co-stimulatory pathway, to disrupt the ability of B7 to bind to CD28 and/or CTLA4, to disrupt the ability of B7 to activate the co-stimulatory pathway, to disrupt the ability of CD80 to bind to CD28 and/or CTLA4, to disrupt the ability of CD80 to activate the co-stimulatory pathway, to disrupt the ability of CD86 to bind to CD28 and/or CTLA4, to disrupt the ability of CD86 to activate the co-stimulatory pathway, and to disrupt the co-stimulatory pathway, in general from being activated. This necessarily includes small molecule inhibitors of CD28, CD80, CD86, CTLA4, among other members of the co-stimulatory pathway; antibodies directed to CD28, CD80, CD86, CTLA4, among other members of the co-stimulatory pathway; antisense molecules directed against CD28, CD80, CD86, CTLA4, among other members of the co-stimulatory pathway; adnectins directed against CD28, CD80, CD86, CTLA4, among other members of the co-stimulatory pathway, RNAi inhibitors (both single and double stranded) of CD28, CD80, CD86, CTLA4, among other members of the co-stimulatory pathway, among other anti-CTLA4 antagonists.

In some embodiments, treatment of a cancer as described herein comprises administering at least one immune checkpoint inhibitor in combination with a TLR7 agonist (e.g., imiquimod, reiquimod, gardiquimod, GS-9620, GS-986). TLR7 agonists from the following families are also contemplated for use with the methods and compositions described herein: (i) imidazoquinolines (e.g., imiquimod, reiquimod, gardiquimod, CL097, 852A), (ii) guanosine analogues (e.g., loxoribine), or (iii) viral or synthetic single-stranded RNAs.

### Pharmaceutically Acceptable Carriers

Therapeutic compositions of the agents disclosed herein can include a physiologically tolerable carrier together with an agent that induces an immune response as described herein, dissolved or dispersed therein as an active ingredient. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without toxicity or the production of undesirable physiological effects such as nausea, dizziness, gastric upset and the like. A pharmaceutically acceptable carrier will not itself promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as topical agents or injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein.

Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. Therapeutic compositions used herein can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

In some embodiments, it can be advantageous to formulate the aforementioned pharmaceutical compositions in dosage unit form for ease of administration and uniformity of dosage. Dosage unit or unitary form refers to physically discrete units suitable as unitary dosages, each unit containing a predetermined quantity of active ingredient calculated to produce the desired therapeutic effect in association with the required pharmaceutical carrier. Examples of such dosage unit forms are tablets (including scored or coated tablets), capsules, pills, powder packets, wafers, injectable solutions or suspensions, teaspoonfuls, tablespoonfuls and the like, and segregated multiples thereof.

### Dosage and Administration

In a treatment method as described herein, an effective amount of an agent that induces an immune response is administered to a patient suffering from or diagnosed as having a tumor (e.g., solid tumor or melanoma). In one aspect, the methods described herein provide a method for treating cancer in a subject. In one embodiment, the subject can be a mammal (e.g., a primate or a non-primate mammal). In another embodiment, the mammal can be a human, although the approach is effective with respect to all mammals. An “effective amount” means an amount or dose generally sufficient to bring about the desired therapeutic or prophylactic benefit in subjects undergoing treatment.

Effective amounts or doses of an immune-inducing reagent for treatment as described herein can be ascertained by routine methods such as modeling, dose escalation studies or clinical trials, and by taking into consideration routine factors, e.g., the mode or route of administration of delivery, the pharmacokinetics of the composition, the severity and course of the disorder or condition, the subject's previous or ongoing therapy, the subject's health status and response to drugs, and the judgment of the treating physician. An exemplary dose for a human is in the range of from about 0.001 to about 8 mg per kg of subject's body weight per day, about 0.05 to 300 mg/day, or about 50 to 400 mg/day, in single or divided dosage units (e.g., BID, TID, QID).

While the dosage range for the composition comprising an agent to induce the immune response depends upon the potency of the composition, and includes amounts large enough to produce the desired effect (e.g., improved tumor treatment), the dosage should not be so large as to cause unacceptable adverse side effects. Generally, the dosage will vary with the formulation (e.g., oral, i.v. or subcutaneous formulations), and with the age, condition, and sex of the patient. The dosage can be determined by one of skill in the art and can also be adjusted by the individual physician in the event of any complication. Typically, the dosage will range from 0.001 mg/day to 400 mg/day. In some embodiments, the dosage range is from 0.001 mg/day to 400 mg/day, from 0.001 mg/day to 300 mg/day, from 0.001 mg/day to 200 mg/day, from 0.001 mg/day to 100 mg/day, from 0.001 mg/day to 50 mg/day, from 0.001 mg/day to 25 mg/day, from 0.001 mg/day to 10 mg/day, from 0.001 mg/day to 5 mg/day, from 0.001 mg/day to 1 mg/day, from 0.001 mg/day to 0.1 mg/day, from 0.001 mg/day to 0.005 mg/day. Alternatively, the dose range will be titrated to maintain serum levels between 0.1 μg/mL and 30 μg/mL.

It is also contemplated herein that the dose of e.g., a checkpoint inhibitor to produce a desired effect can be reduced when administered in combination with e.g., ablative FP and imiquimod compared to the dose that is administered for conventional treatment of the cancer (e.g., melanoma).

Administration of the doses recited above can be repeated for a limited period of time or as necessary. In some embodiments, the doses are given once a day, or multiple times a day, for example but not limited to three times a day. In one embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Agents useful in the methods and compositions described herein depend on the site of the tumor and can be administered topically, intravenously (by bolus or continuous infusion), intratumorally, orally, by inhalation, intraperitoneally, intramuscularly, subcutaneously, intracavity, and can be delivered by peristaltic means, if desired, or by other means known by those skilled in the art. For the treatment of certain cancers (e.g., metastatic disease), the agent can be administered systemically.

Therapeutic compositions containing at least one agent can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

Combination Therapy: Provided herein are methods for treating cancer, comprising administering a combination of at least two different agents (e.g., 2, 3, 4, 5, 6, 7, 8, 9, or 10 different agents). In one embodiment, the combination therapy comprises administration of at least one immune checkpoint inhibitor with at least one endosomal TLR agonist (e.g., an agonist of TLR3, TLR7, TLR8 or TLR9). In another embodiment, the combination therapy comprises administration of at least one immune checkpoint inhibitor in combination with a fractional laser therapy treatment. In another embodiment, the combination therapy comprises administration of at least one immune checkpoint inhibitor, at least one endosomal TLR agonist (e.g., an agonist of TLR3, TLR7, TLR8 or TLR9) and at least one fractional laser therapy treatment. In another embodiment, the combination therapy comprises administration of at least one immune checkpoint inhibitor, at least one endosomal TLR agonist (e.g., an agonist of TLR3, TLR7, TLR8 or TLR9), at least one fractional laser therapy treatment and a CTLA-4 inhibitor (e.g., an antibody against CTLA-4).

When at least two agents are administered as a combination therapy, they can be administered simultaneously. In other embodiments, the at least two agents are administered separately or concurrently. The agents can be delivered in any desired order by one of skill in the art. The immune checkpoint inhibitors can be administered intratumorally, systemically, orally or by any other desired forms of administration. Endosomal TLR agonists are contemplated for delivery by intratumoral injection, injection into a tumor's blood supply or by topical administration.

In one embodiment, the anti-tumor response to combination therapy as described is synergistic.

### Efficacy Measurement

The efficacy of a treatment comprising an agent that induces an immune response (e.g., a local intratumoral immune response, reduction in tumor or lesion size, improved sensitivity to treatment with a checkpoint inhibitor etc.) can be determined by the skilled clinician. However, a treatment is considered “effective treatment,” as the term is used herein, if any one or all of the signs or symptoms of, as but one example, cancer are altered in a beneficial manner, other clinically accepted symptoms or markers of disease are improved or ameliorated, e.g., by at least 10% following treatment with an inhibitor. Efficacy can also be measured by failure of an individual to worsen as assessed by hospitalization or need for medical interventions (e.g., progression of the disease is halted or at least slowed). Efficacy in a population of patients can also be determined by measuring mortality rates due to advanced metastatic disease. Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the progression of the cancer; or (2) relieving the disease, e.g., causing regression of symptoms; and (3) preventing or reducing the likelihood of the development of metastases, including metastatic melanoma.

The present invention may be as defined in any of the following numbered paragraphs.

1. A method for treating cancer in a subject, the method comprising: (a) administering at least one drug to a subject having a tumor, and (b) contacting tissue of the tumor with a fractional laser, thereby treating cancer in the subject.

2. The method of paragraph 1, wherein the at least one drug is administered systemically.

3. The method of paragraph 1, wherein the at least one drug is an immune checkpoint inhibitor.

4. The method of paragraph 3, wherein the immune checkpoint inhibitor is an inhibitor of PD1, PDL1, TIM-3, or CTLA4.

5. The method of paragraph 3, wherein the immune checkpoint inhibitor is ipilimumab, tremelimumab, nivolumab, or pembrolizumab.

6. The method of paragraph 1, wherein the at least one drug is administered locally.

7. The method of paragraph 6, wherein the at least one drug is administered topically or injected into the tumor tissue.

8. The method of paragraph 6, wherein the at least one drug is an agonist of TLR3, TLR7, TLR8 or TLR9.

9. The method of paragraph 8, wherein the TLR7 agonist is imiquimod, reiquimod, or gardiquimod.

10. The method of paragraph 1, further comprising administering at least two drugs.

11. The method of paragraph 10, wherein the at least two drugs comprise imiquimod and at least one immune checkpoint inhibitor.

12. The method of paragraph 1, wherein the step of administering a drug to the subject is performed at least twice.

13. The method of paragraph 1, wherein the step of contacting tumor tissue with the fractional laser is performed at least twice.

14. The method of paragraph 1, wherein the administering step and the contacting step are performed simultaneously.

15. The method of paragraph 1, wherein the administering step is performed before or after the contacting step.

16. The method of paragraph 1, wherein the cancer is melanoma or pancreatic cancer.

17. The method of paragraph 1, wherein the fractional laser is a CO₂ laser.

18. The method of paragraph 1, wherein the fractional laser penetrates to a depth of at least 0.1 mm into the tumor tissue.

19. The method of paragraph 1, wherein treatment with the fractional laser induces a local immune response in the tumor tissue.

20. The method of paragraph 1, wherein treatment with the fractional laser does not damage the stratum corneum.

21. The method of paragraph 1, wherein treatment with the fractional laser does not induce scarring or crusting of the tumor tissue.

22. The method of paragraph 1, wherein the area of treatment comprises at least 0.25 mm.

23. The method of paragraph 1, wherein the energy of the fractional laser is 1 mJ to 200 mJ.

24. The method of paragraph 23, wherein 50 mJ or 100 mJ of energy is used for a superficial lesion and 200 mJ of energy is used for a deep tumor.

25. The method of paragraph 1, wherein the pulse duration of the fractional laser is 100 usec to 10 msec.

26. The method of paragraph 25, wherein the pulse duration of the fractional laser is 2 msec.

27. The method of paragraph 1, wherein the spot size of the fractional laser is 10 um to 1 mm.

28. The method of paragraph 1, wherein the penetration depth of the fractional laser is ⅓ the depth of the tumor.

29. A method of promoting resistance of a subject to recurrence of a cancer, the method comprising: (a) administering at least one drug to a subject having a tumor, and (b) contacting tissue of the tumor with a fractional laser, thereby promoting resistance of the subject to a recurrence of the cancer.

30. The method of paragraph 29, wherein the at least one drug is administered systemically.

31. The method of paragraph 30, wherein the at least one drug is an immune checkpoint inhibitor.

32. The method of paragraph 31, wherein the immune checkpoint inhibitor is an inhibitor of PD1, PDL1, TIM-3, or CTLA4.

33. The method of paragraph 31, wherein the immune checkpoint inhibitor is ipilimumab, tremelimumab, nivolumab, or pembrolizumab.

34. The method of paragraph 29, wherein the at least one drug is administered locally.

35. The method of paragraph 29, wherein the at least one drug is administered topically or injected into the tumor tissue.

36. The method of paragraph 34, wherein the at least one drug is an agonist of TLR3, TLR7, TLR8 or TLR9.

37. The method of paragraph 36, wherein the TLR7 agonist is imiquimod, reiquimod, or gardiquimod.

38. The method of paragraph 29, further comprising administering at least two drugs.

39. The method of paragraph 38, wherein the at least two drugs comprise imiquimod and at least one immune checkpoint inhibitor.

40. The method of paragraph 29, wherein the step of administering a drug to the subject is performed at least twice.

41. The method of paragraph 29, wherein the step of contacting tumor tissue with the fractional laser is performed at least twice.

42. The method of paragraph 29, wherein the administering step and the contacting step are performed simultaneously.

43. The method of paragraph 29, wherein the administering step is performed before or after the contacting step.

44. The method of paragraph 29, wherein the cancer is melanoma or metastatic melanoma.

45. The method of paragraph 29, wherein the fractional laser is a CO2 laser.

46. The method of paragraph 29, wherein the fractional laser penetrates to a depth of at least 0.1 mm into the tumor tissue.

47. The method of paragraph 29, wherein treatment with the fractional laser induces a local immune response in the tumor tissue.

48. The method of paragraph 29, wherein treatment with the fractional laser does not damage the stratum corneum.

49. The method of paragraph 29, wherein treatment with the fractional laser does not induce scarring or crusting of the tumor tissue.

50. The method of paragraph 29, wherein the area of treatment comprises at least 0.25 mm2.

51. The method of paragraph 29, wherein the energy of the fractional laser is 1 mJ to 200 mJ.

52. The method of paragraph 51, wherein 50 mJ of energy is used for a superficial lesion and 200 mJ of energy is used for a deep tumor.

53. The method of paragraph 51, wherein the energy of the fractional laser is 100 mJ.

54. The method of paragraph 29, wherein the pulse duration of the fractional laser is 100 usec to 10 msec.

55. The method of paragraph 54, wherein the pulse duration of the fractional laser is 2 msec.

56. The method of paragraph 29, wherein the spot size of the fractional laser is 10 um to 1 mm.

57. The method of paragraph 29, wherein the penetration depth of the fractional laser is ⅓ the depth of the tumor.

### EXAMPLES

### Example 1: Rescuing Response to Immune Checkpoint Blockade in Neoantigen-Deficient Cancers

Immune checkpoint inhibitors targeting the cytotoxic T lymphocyte-associated antigen-4 (CTLA-4)1 and programmed cell death-1 (PD-1) pathways2,3 can deliver durable anti-tumor effects. However, a major fraction of patients with metastatic melanoma and other cancers fail to respond to checkpoint blockade therapy4. Recent studies indicate that efficacy of checkpoint blockade correlates with pre-treatment or treatment-induced T cell infiltration and higher burdens of tumor-specific neoantigens5-12. The preponderance of ultraviolet radiation (UVR)-induced somatic mutations in melanoma has been proposed to play an important role in responses to immunotherapies. However, responsiveness to checkpoint inhibitors is also associated with development of vitiligo, which is reported in ˜25% of patients with melanoma but not other cancers undergoing anti-PD-1 therapy13. The association of melanoma-associated vitiligo with significantly higher rates of objective tumor response to anti-PD-1 suggests that evolution towards immune recognition of wildtype melanocytic antigens might be beneficial. Here, a BrafV600E/Pten−/− syngeneic mouse melanoma model was used to first test whether efficacy of checkpoint blockade is modulated by presence of UVR-associated neoantigens. It was observed that melanoma clones bearing numerous UVB-induced mutations were markedly more inflamed and responsive to PD-1 inhibition than their matched parental melanomas. To “rescue” responsiveness to checkpoint blockade in neoantigen-deficient tumors, checkpoint inhibitors were combined with topical imiquimod, a Toll-like receptor (TLR) 7 agonist, plus ablative fractional photothermolysis (aFP), a laser method commonly used for treating scarring and photoaging14. In resistant models of melanoma and pancreatic adenocarcinoma, addition of imiquimod and aFP to anti-PD-1 produced both local and systemic/distant tumor regressions with long-term survival in 50-60% of cases. This combination therapy stimulated expansion of CD8+ T cells specific for wildtype melanocytic antigen recognition and protected against engraftment of unrelated B16 melanoma in long-term melanoma survivors. In addition, combination treatment of UVB-mutation-bearing melanomas conferred lasting immunity even against mutation deficient melanomas, consistent with a mechanism of epitope spreading towards shared melanocytic antigens. These results demonstrate the functional importance of mutational load and neoantigens in anti-tumor immunity. Taken together with human data on treatment-associated vitiligo13, they also indicate that therapeutic strategies which enhance responses against wildtype tumor-lineage self-antigens, such as the novel combination of imiquimod, aFP, and checkpoint inhibitors, can bypass the requirement for neoantigens and produce major regressions of non-immunogenic tumors.

Recent studies have identified neoantigen-reactive T cells in mouse models of sarcoma15 and patients with melanoma16-20 and cholangiocarcinoma21. However, the proportion of non-synonymous mutations encoding neoantigens for which specific T cells can be detected is low in many tumor types, recurrent classes of neoantigens associated with response are not found in all cohorts, and neoantigen burden does not predict clinical benefit for individual patients8,11. Understanding the contribution of neoantigens to anti-tumor immunity has been limited by the uniqueness of mutational landscapes across patient tumors, variation in human immune responses, and environmental factors such as composition of the intestinal microbiome22,23.

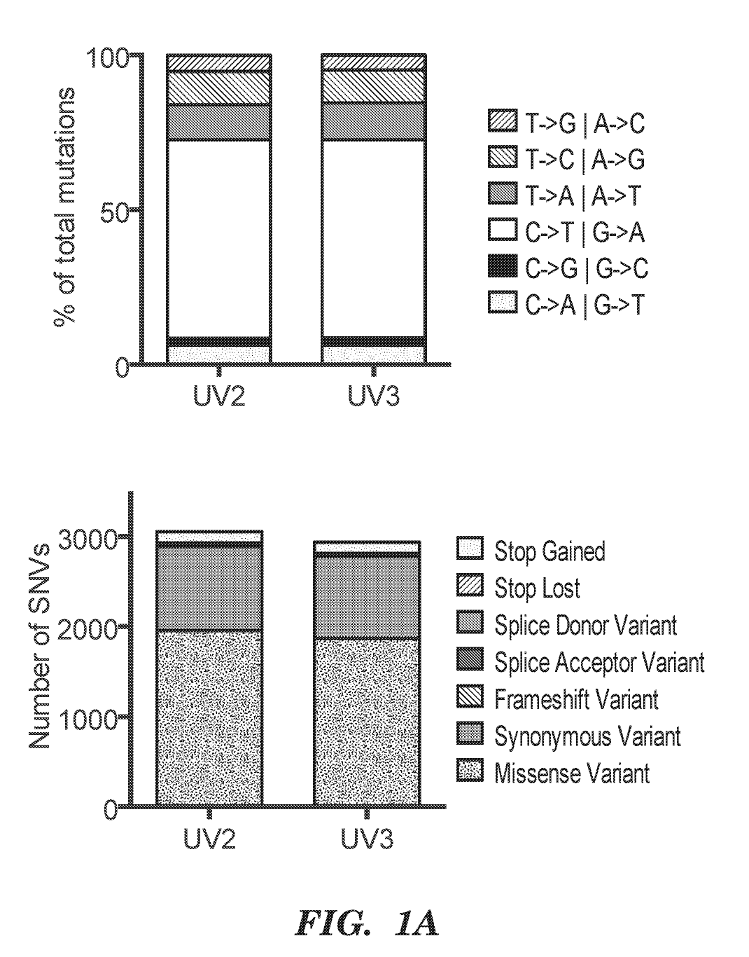

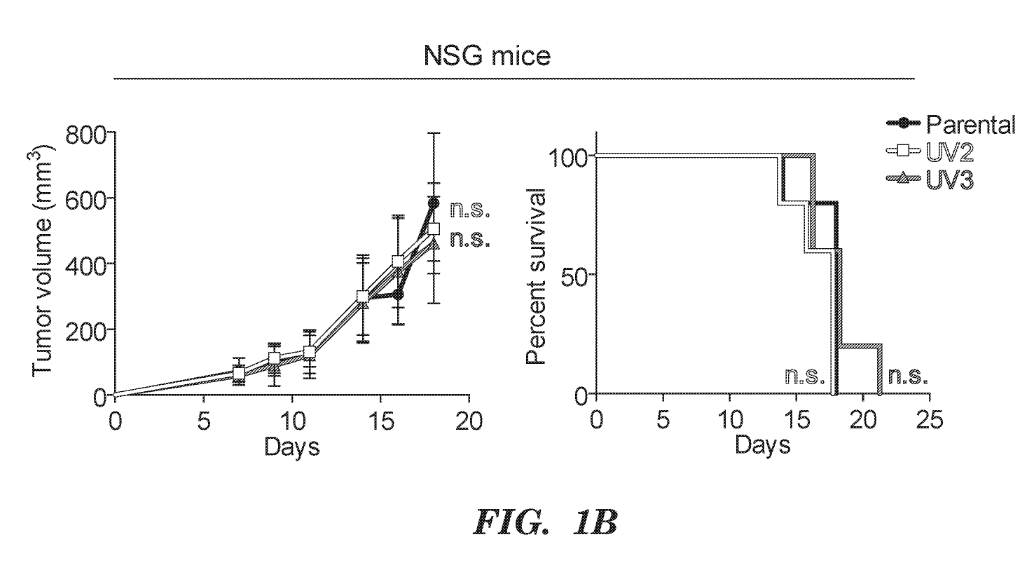

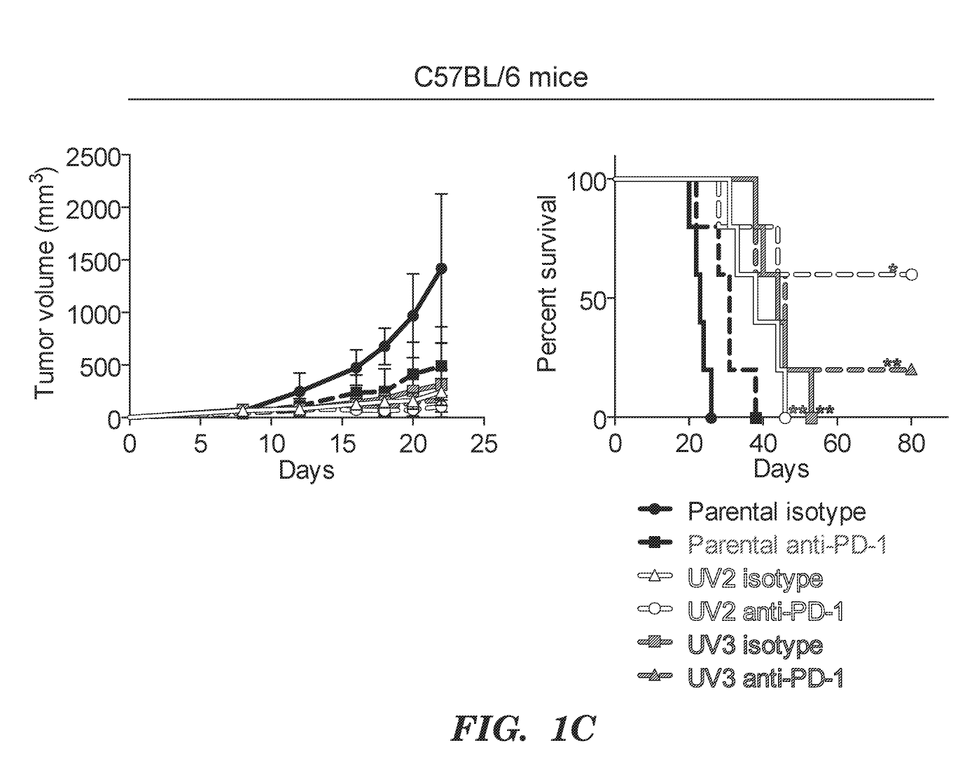

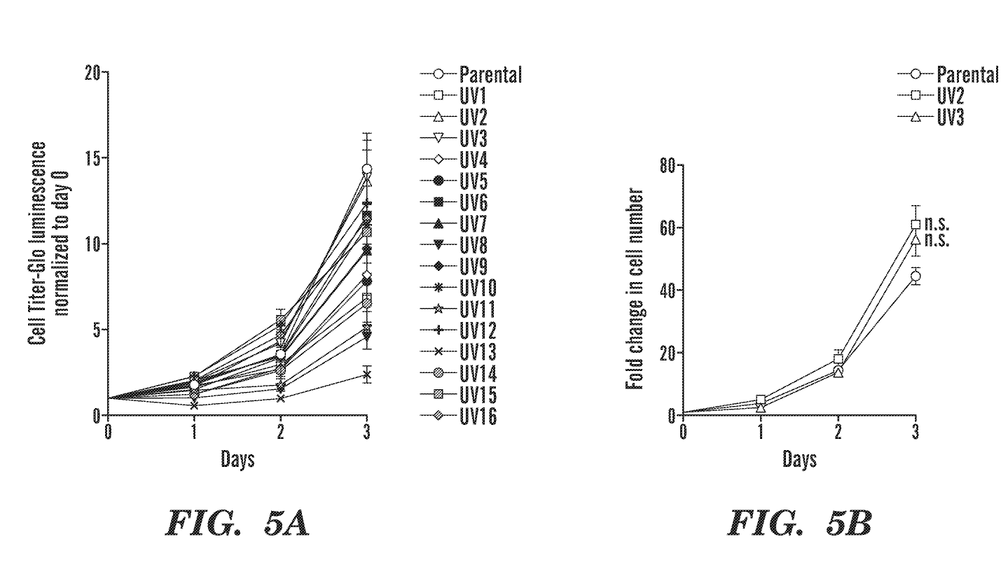

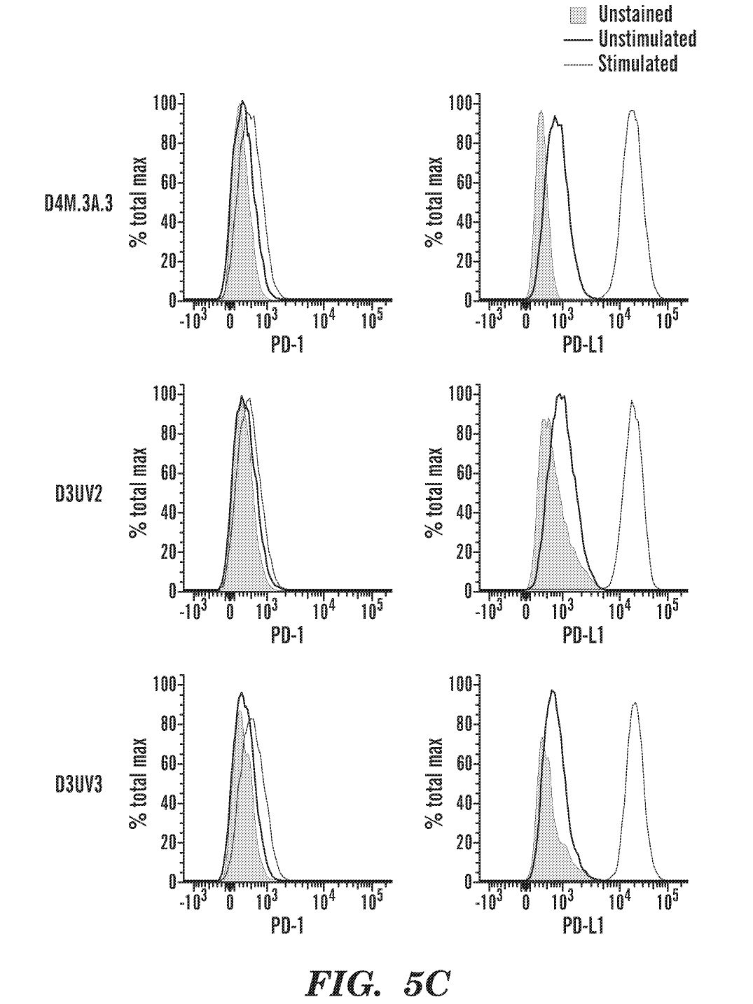

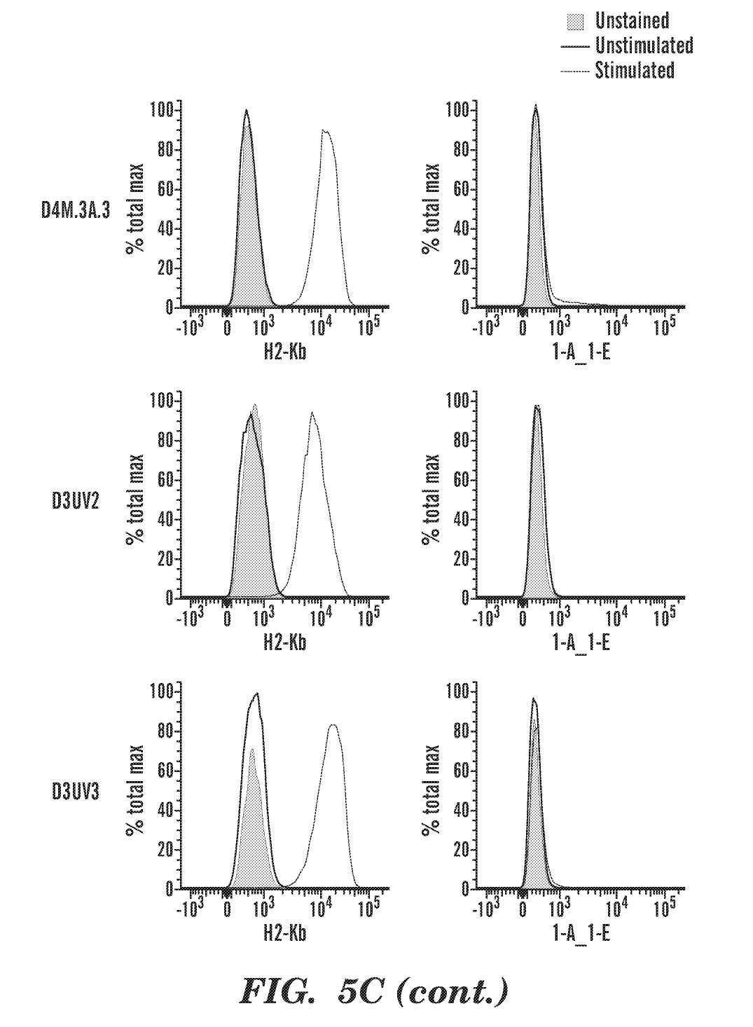

To study the role of tumor-specific neoantigens in response to checkpoint blockade, a transplantable mouse melanoma model was developed based on the poorly immunogenic D4M.3A melanoma cell line24 established from a Tyr:CreER;BrafCA;Ptenlox/lox mouse25 fully backcrossed to the C57BL/6 background. A stable cell line (“parental”) was derived from a single cell clone of D4M.3A. To mimic the mutagenic effects of sun exposure, the leading environmental risk factor for skin cancer, this parental melanoma was subjected to UVB irradiation in vitro and a series of single cell “UV clones” were isolated. Two clones, D3UV2 (“UV2”) and D3UV3 (“UV3”), had the same in vitro growth kinetics and expression of PD-L1, PD-1, and MHC class I and II as the parental cell line (FIG. 5A-5C). Compared to the parental cell line, UV2 and UV3 contain an additional 79 and 87 mutations/Mb, respectively, which is comparable to somatic mutation rates in human melanomas that range across 0.1-100/Mb26. As expected, most mutations resulted from C>T transitions associated with UVB mutagenesis and occurred at a 2:1 ratio of non-synonymous to synonymous events (FIG. 1A).

Consistent with proliferation rates in culture, there was no significant difference in tumor growth kinetics in immunodeficient NOD/SCID/γ-chain-null (NSG) mice following subcutaneous inoculation of parental or UVB-mutagenized cells (FIG. 1B). In immunocompetent (syngeneic) C57BL/6 hosts, UV2 and UV3 tumors also engrafted readily (FIG. 1C). However, in contrast to parental melanomas, survival of mice with UV clone tumors was markedly improved by anti-PD-1, with stable complete clearance of 20-60% of these tumors versus 0% of parental melanomas (FIG. 1C).

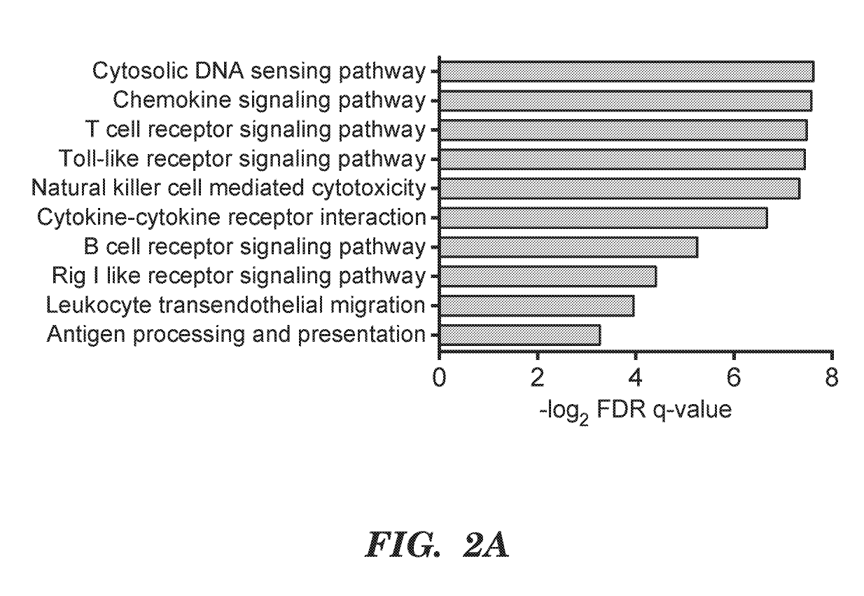

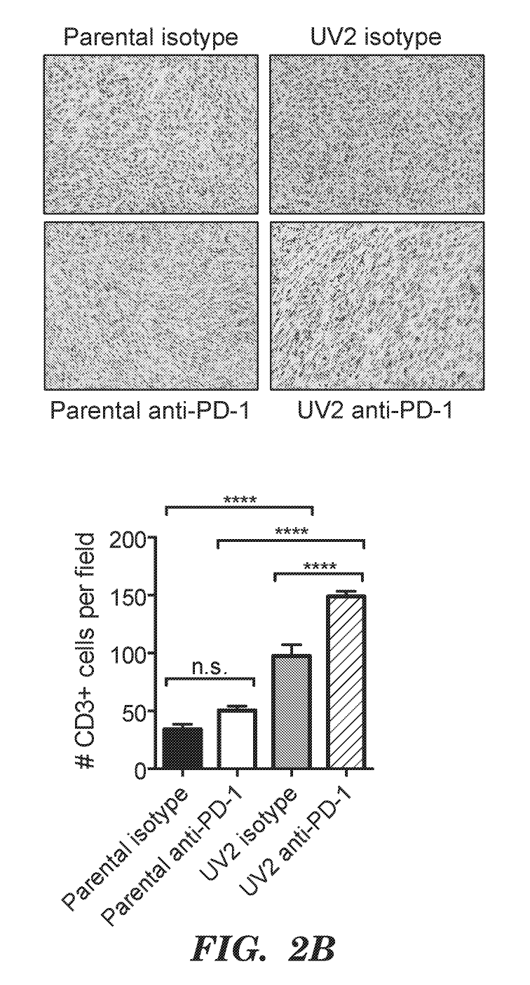

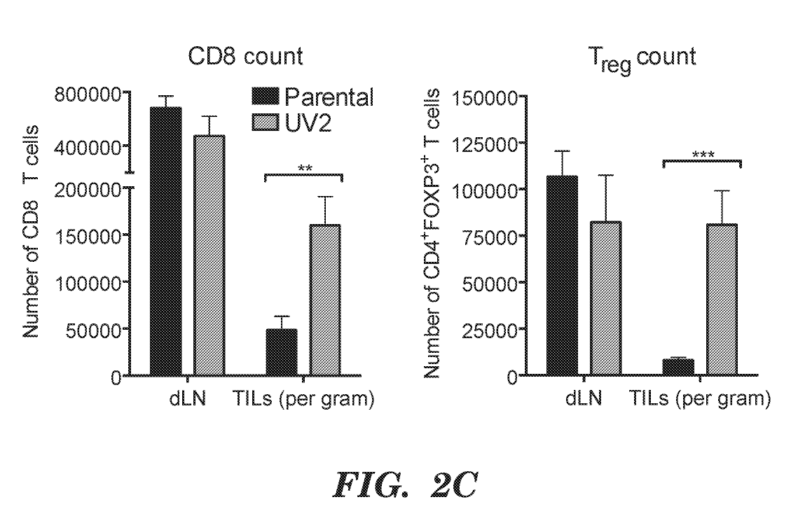

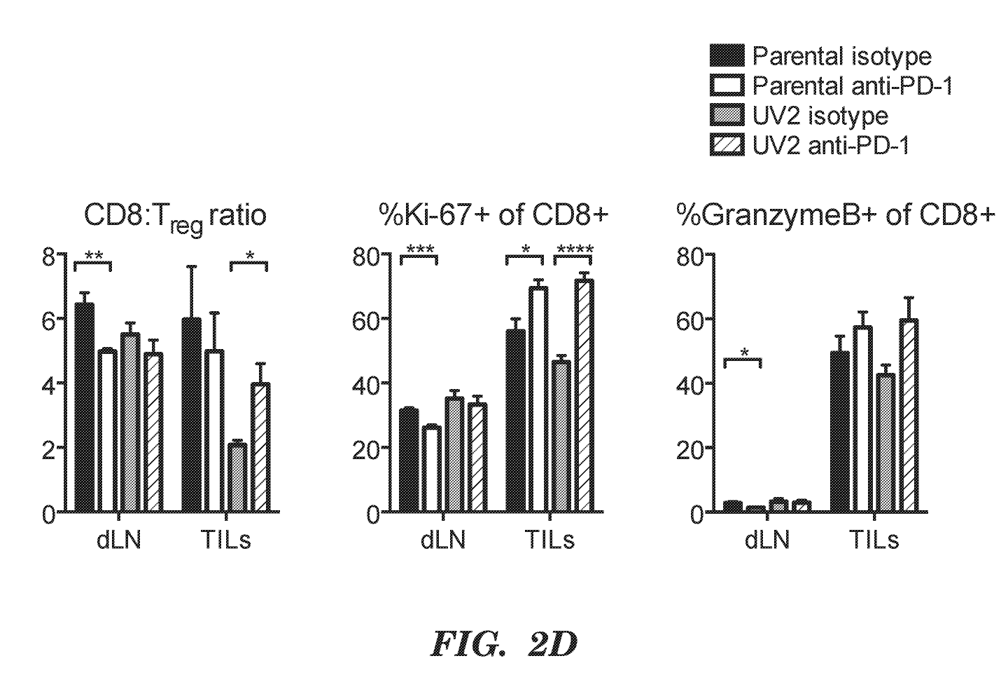

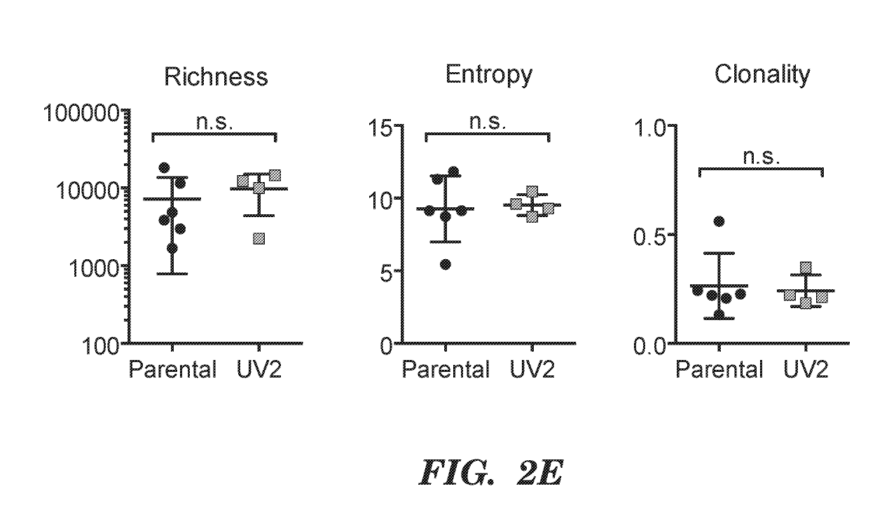

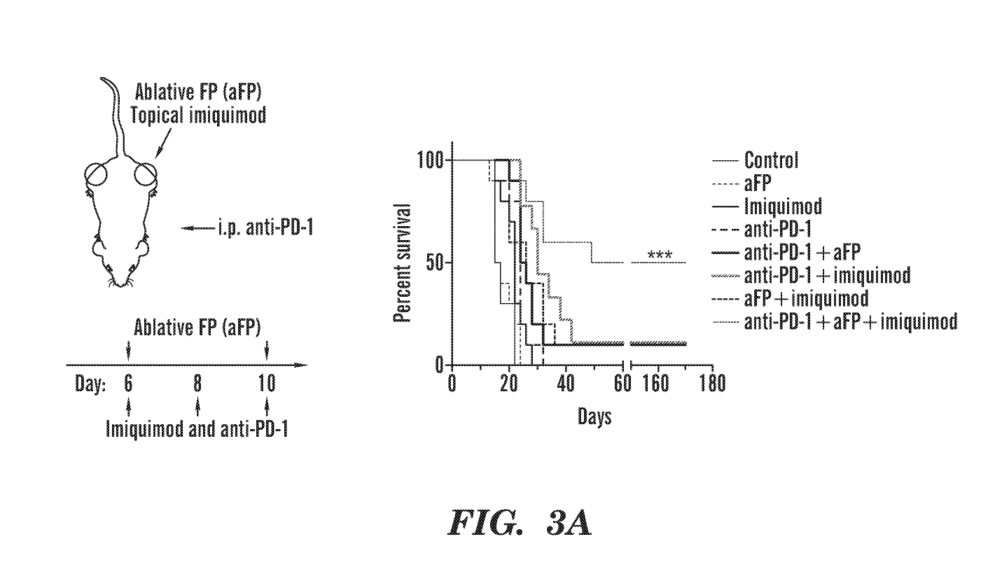

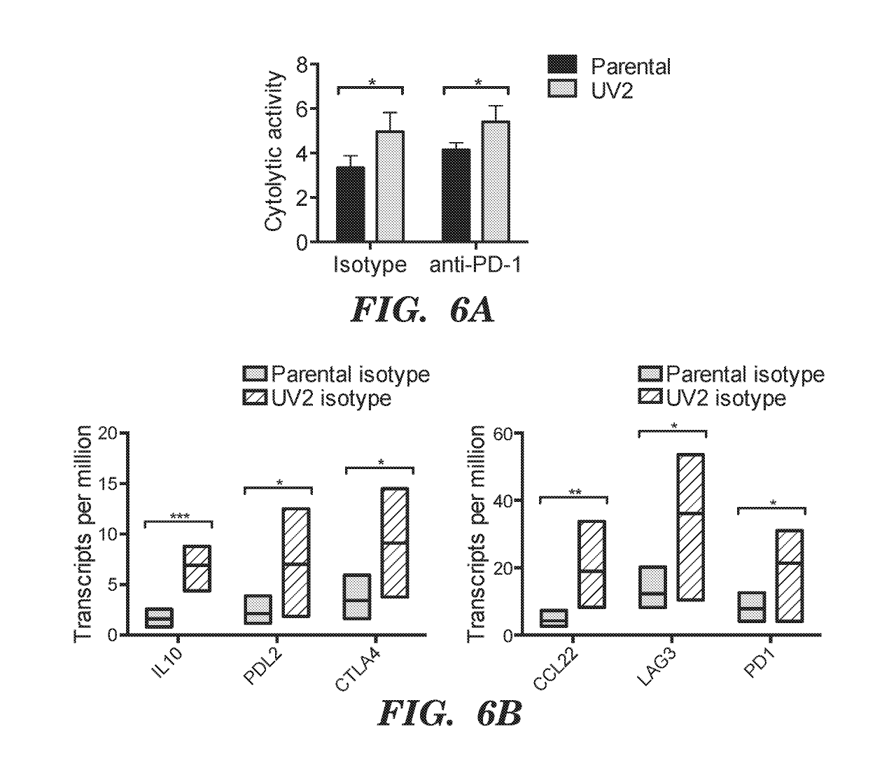

To understand how neoantigens affect the tumor microenvironment, RNA-sequencing of whole tumors was performed. Gene set enrichment analysis (GSEA)27 revealed strong enrichment of multiple immune-associated gene sets in UV2 melanomas compared to parental melanomas, extending across innate and adaptive immunity (FIG. 2a, Table 1). Immunohistochemical analysis confirmed significantly higher numbers of tumor-infiltrating T cells in UV2 melanomas than in parental melanomas (FIG. 2B). UV2 tumors contained significantly higher numbers of CD8+ T cells (FIG. 2C) and had correspondingly greater immune cytolytic activity28 (FIG. 6A). However, this was accompanied by T cell dysfunction, with a decrease in Ki67+ CD8+ T cells, greater numbers of CD4+FOXP3+ Treg cells, lower CD8:Treg ratio, and increased expression of inhibitory receptors and molecules (FIGS. 2C, 2D, & 6B). Treatment of UV2 tumors with anti-PD-1 restored intratumoral CD8:Treg ratio and increased the proportion of CD8+ T cells positive for Ki67 and granzyme B (FIG. 2D). In contrast, in parental melanomas anti-PD-1 treatment did not improve the CD8:Treg ratio and had a smaller effect on Ki67+ and granzyme B+ CD8+ T cell populations (FIG. 2D). T cell receptor (TCR) β-chain sequencing of tumor infiltrating lymphocytes (TILs) demonstrated no change in richness, clonality, or diversity of TCR clonotypes (FIG. 2E), indicating that the presence of neoantigens can provoke responses of multiple CD8+ T cell clones without emergence of one or a few dominant clones.

These results support human bioinformatics analyses that have demonstrated greater efficacy of checkpoint blockade in patient populations with higher predicted neoantigen loads8-10,12. In addition, the observation of a more inflamed tumor microenvironment in UV2 melanomas is consistent with multiple studies showing that responses to checkpoint inhibitors are associated with pre-existing T cell infiltration into tumors5-7,11.

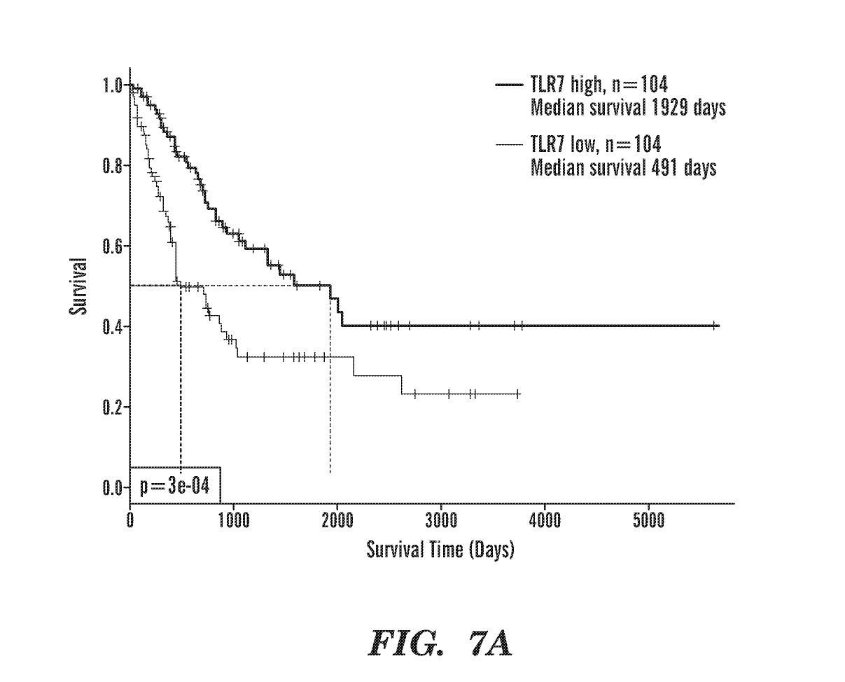

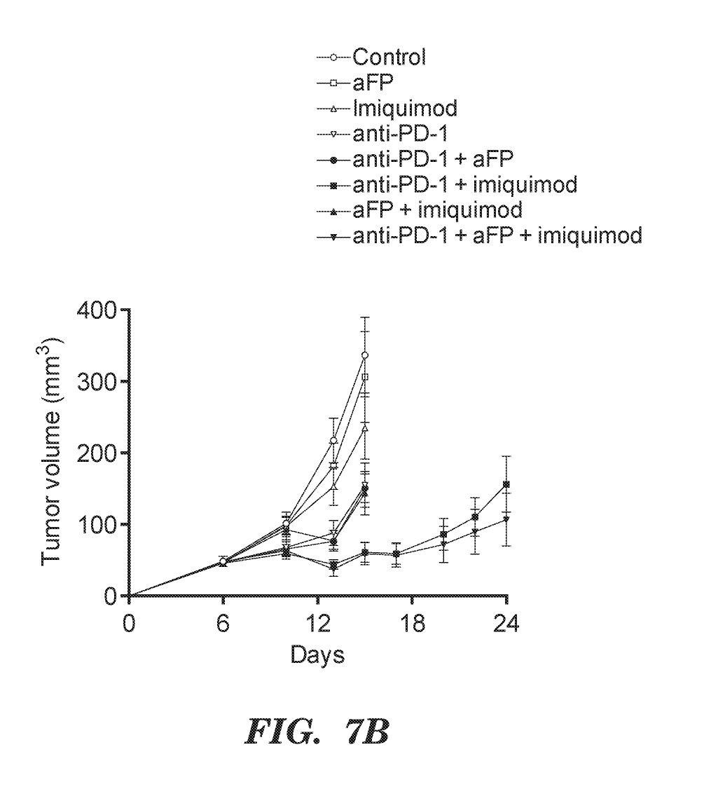

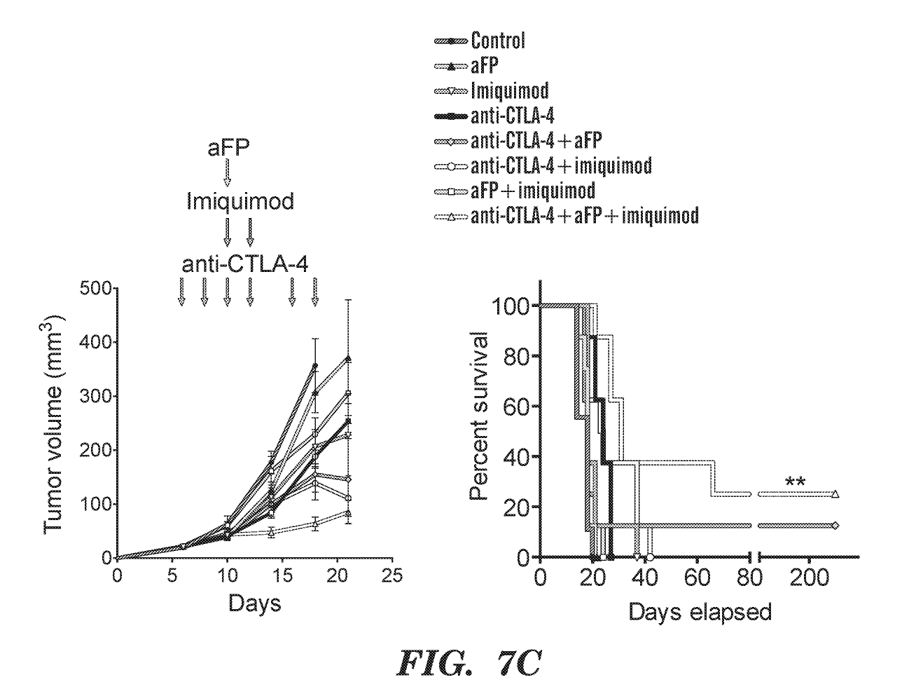

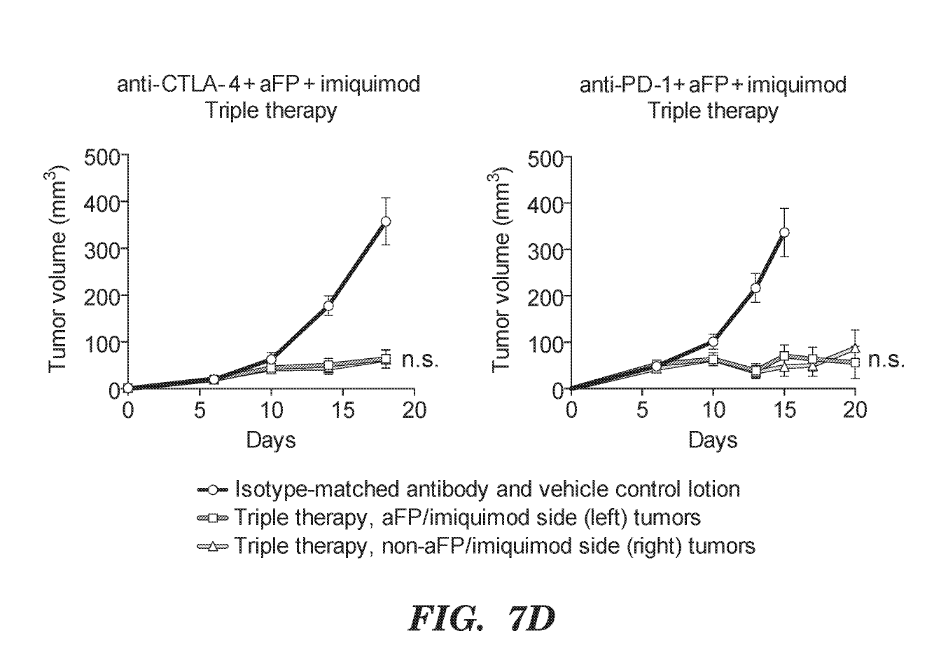

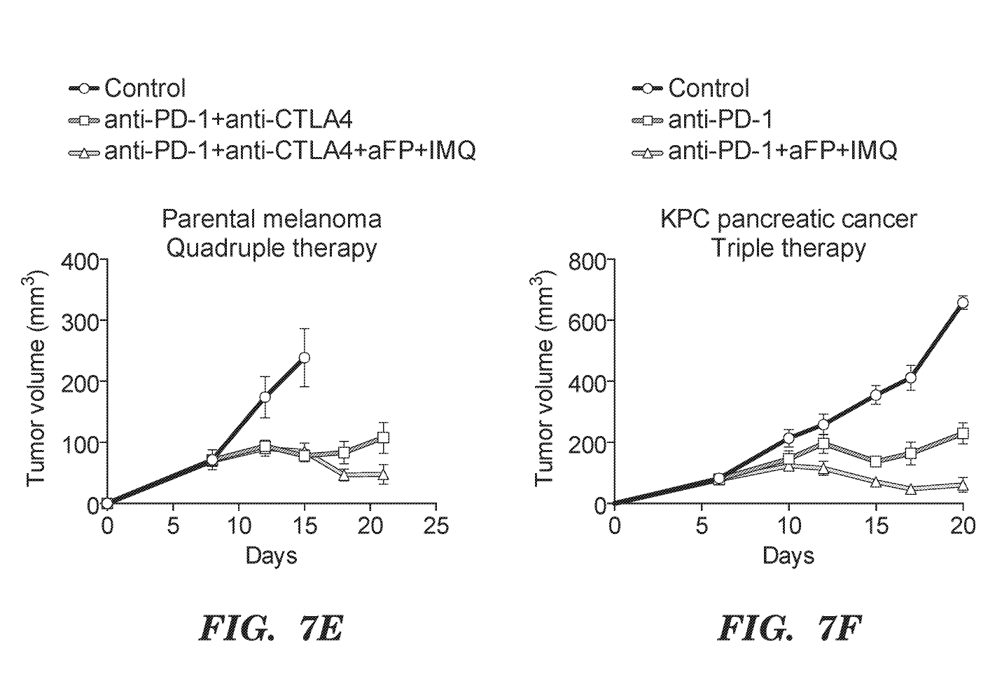

Next, attention was focused on the parental melanoma model, which recapitulates poorly inflamed human tumors that have lower mutational loads and fail anti-PD-1 therapy. For patients with non-inflamed, neoantigen-deficient tumors, enhancement of inflammation in the tumor microenvironment might improve responses to checkpoint blockade. To induce local inflammation, the combination of topical imiquimod and ablative fractional photothermolysis (aFP) was tested. Imiquimod, which induces pro-inflammatory cytokines including type I interferons (IFNs), is clinically used for treating basal cell carcinoma, actinic keratoses, and lentigo maligna melanoma29,30. Higher expression of TLR7 is associated with longer survival in melanoma patients (FIG. 7A). AFP was chosen because it creates numerous microscopic columns of thermal injury with intact interspersed tissue 14 and can thus produce partial tumor ablation while sparing many tumor-infiltrating immune cells31. AFP parameters were adjusted to ablate only ˜2.4% of subcutaneous tumors, thereby aiming to enhance inflammation without elimination of intratumoral immune cell populations.

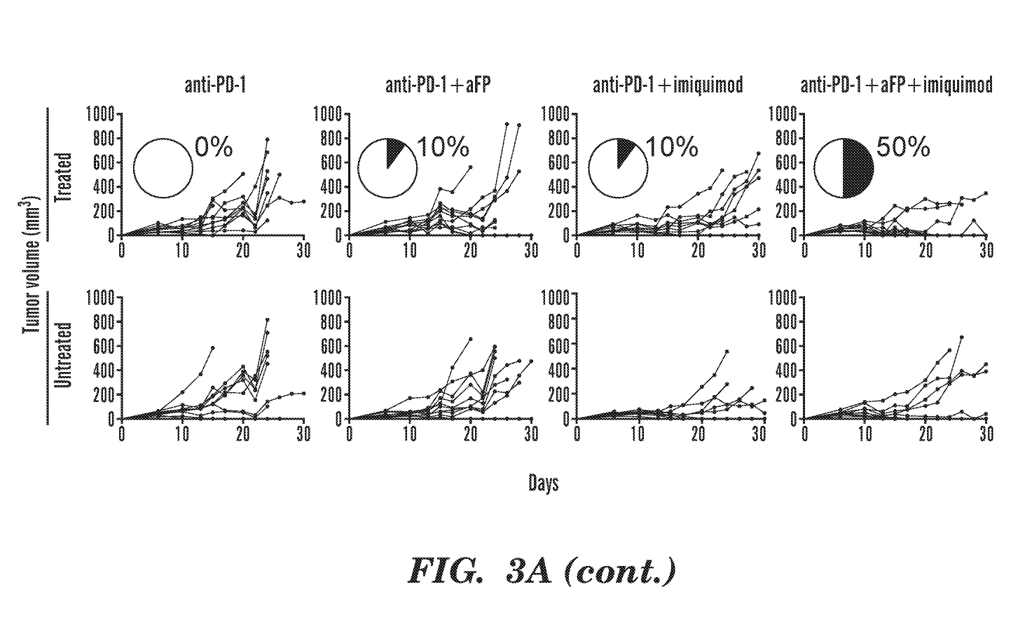

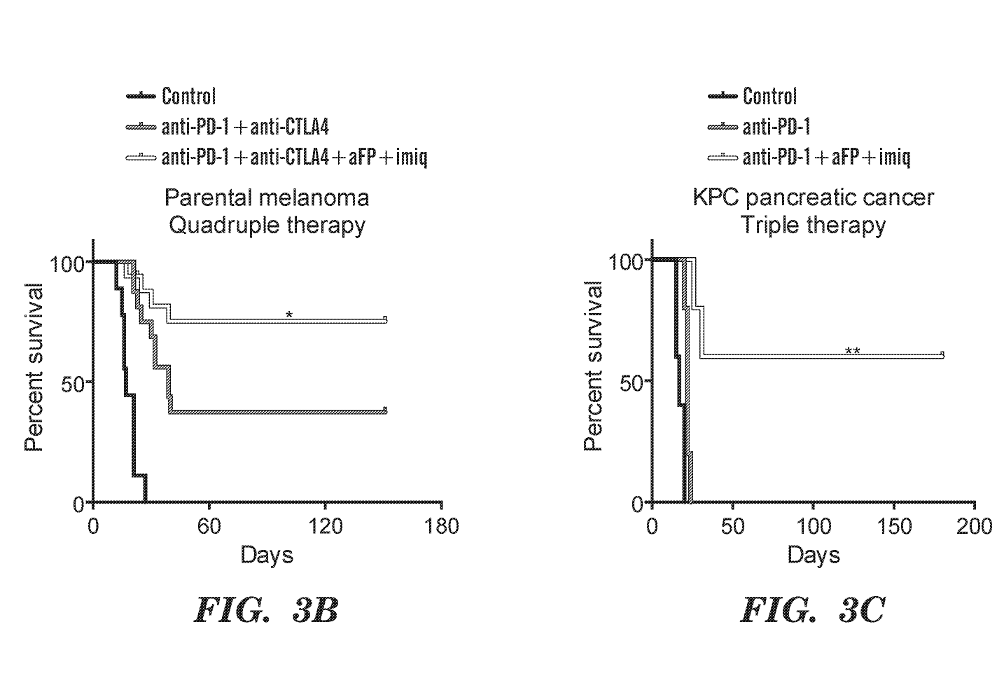

To evaluate combinatorial efficacy with immune checkpoint blockade, mice with bilateral flank parental melanomas were treated with all combinations of imiquimod, aFP, and/or anti-PD-1 (FIG. 3A & 7B). AFP and topical imiquimod were applied to only one tumor per mouse while anti-PD-1 was administered systemically. Complete response rates, with complete regression of both tumors, improved from 0% with any single agent therapy to 10% with any combination of two treatments, to 50% following the triple combination of imiquimod+aFP+anti-PD-1 (FIG. 3A, FIG. 7B). Combinatorial efficacy of triple therapy with anti-CTLA-4 instead of anti-PD-1 imiquimod+aFP+anti-CTLA-4) was also observed, with complete responses in 25% of mice (FIG. 7C). Virtually identical growth reduction was observed in tumors on both mouse flanks despite unilateral imiquimod+aFP treatments (FIG. 3D), indicating that local administration of imiquimod and aFP mediates an abscopal effect against neoantigen-deficient parental melanomas when combined with checkpoint inhibition.

Consistent with the complementary activity and recent clinical success of dual PD-1 and CTLA-4 blockade in metastatic melanoma32,33, responses to anti-PD-1+anti-CTLA-4 in mice with parental melanomas were superior to either alone (FIG. 3B). Addition of imiquimod and aFP further increased the complete bilateral response rate to 75% (FIG. 3B, FIG. 7E), demonstrating that imiquimod and aFP are also synergistic with dual checkpoint blockade. Additionally, triple therapy was tested as a treatment for pancreatic ductal adenocarcinoma, which has been refractory to checkpoint blockade in clinical trials, using the transplantable syngeneic KPC mouse model (KrasLSL.G12D; p53R172H;Pdx1:Cre). While PD-1 monotherapy provided no benefit, triple therapy induced bilateral pancreatic tumor regressions with durable complete responses in 60% of mice (FIG. 3C, FIG. 7F).

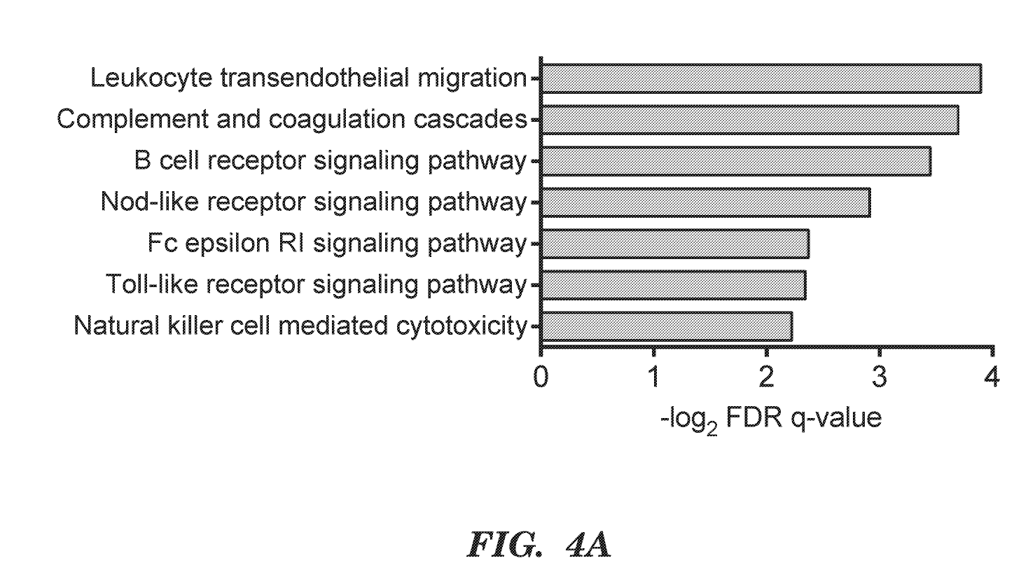

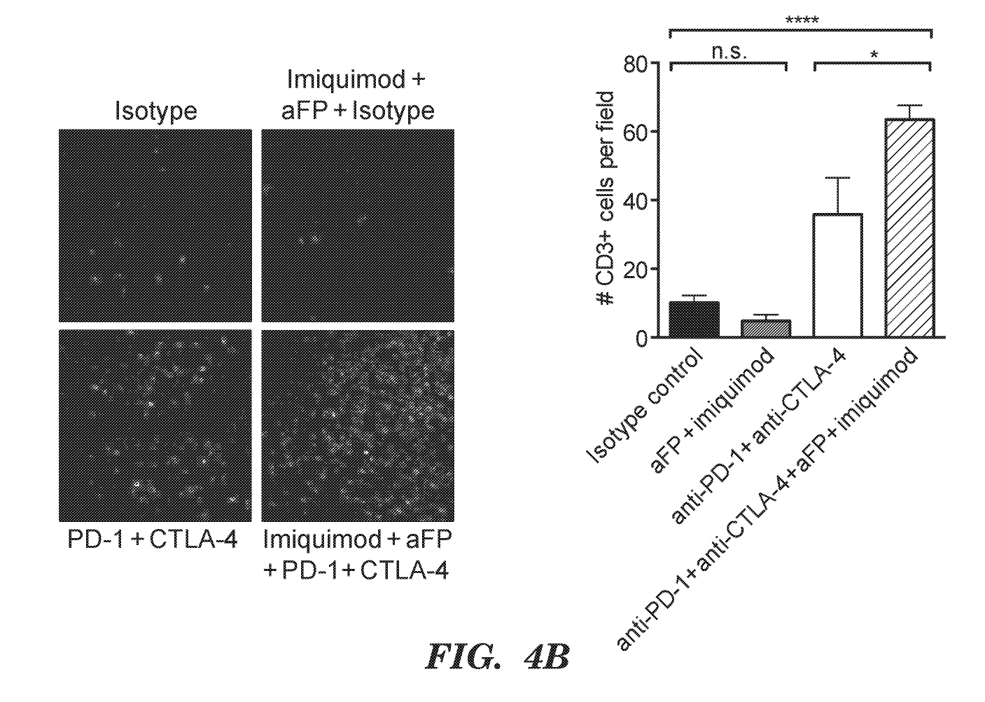

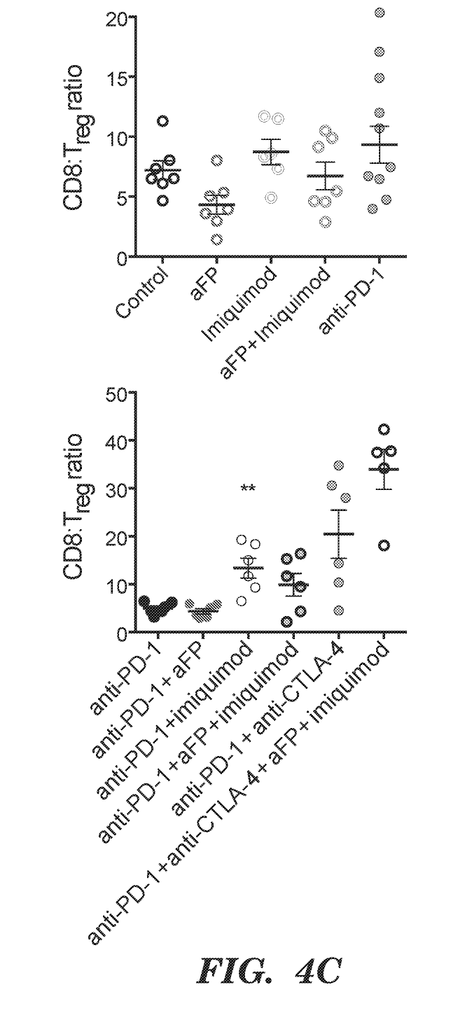

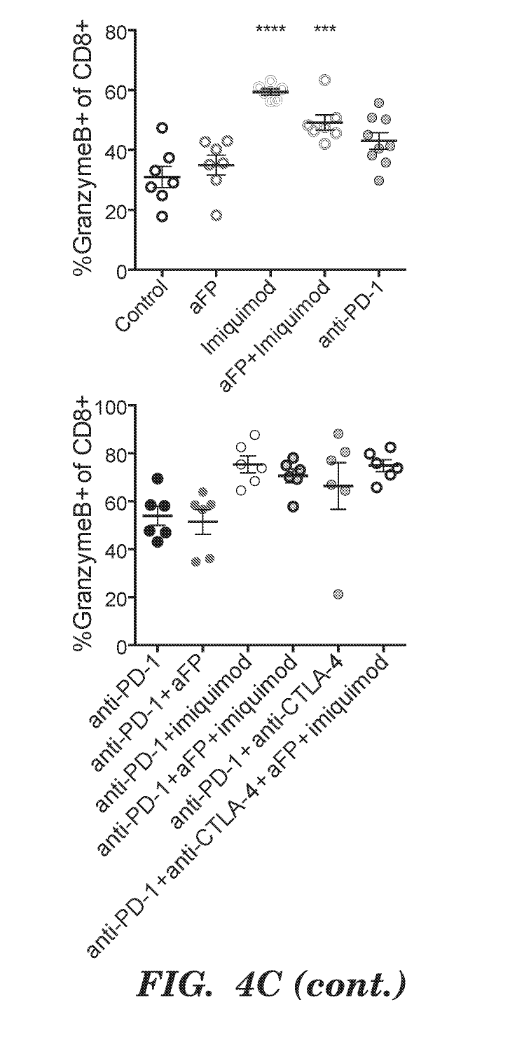

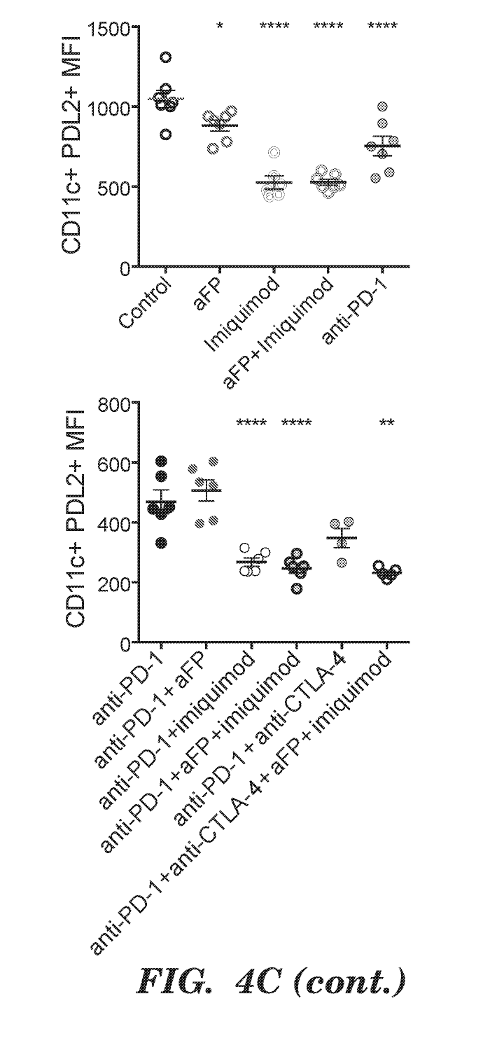

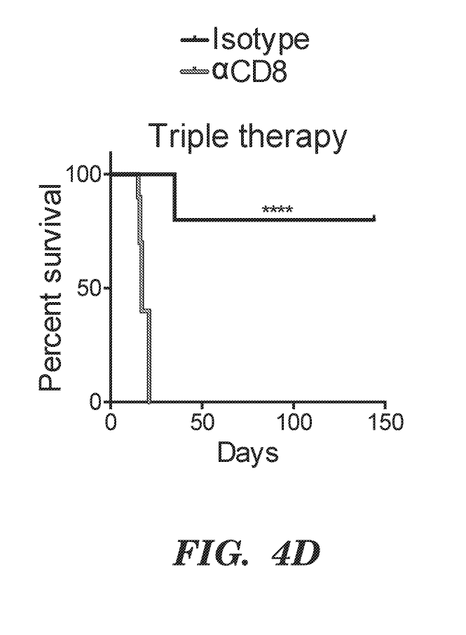

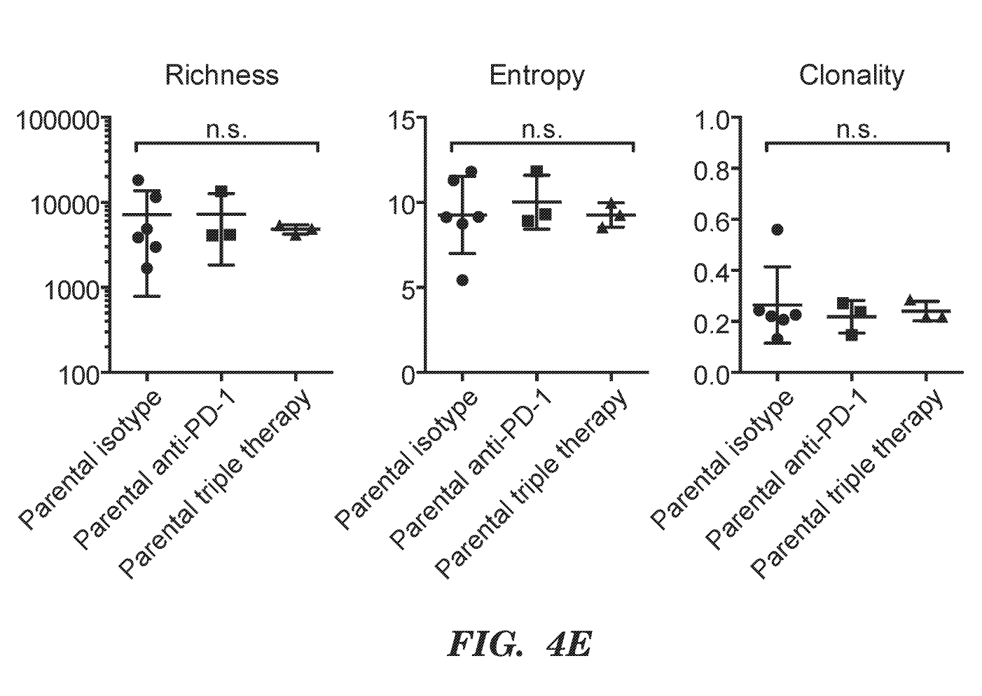

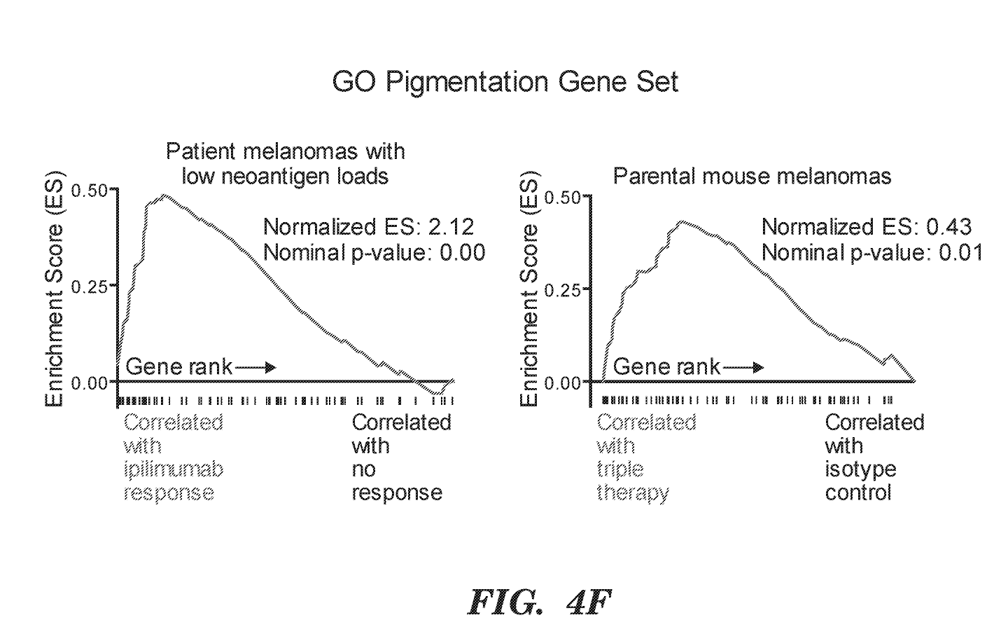

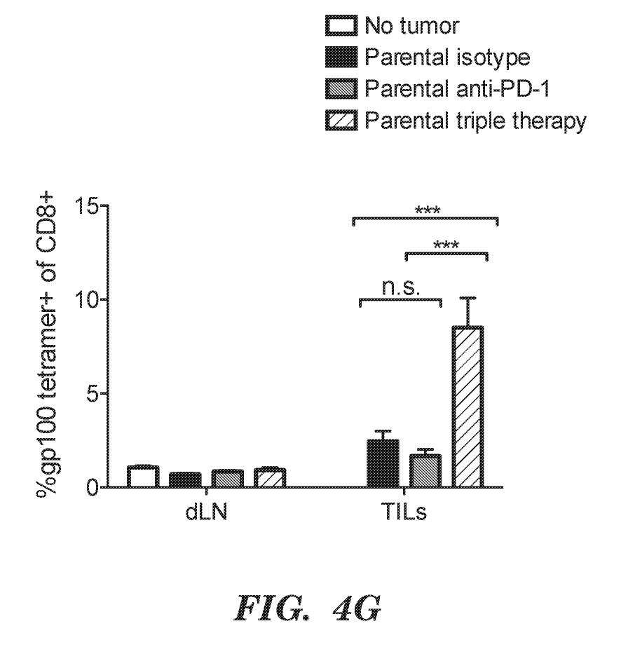

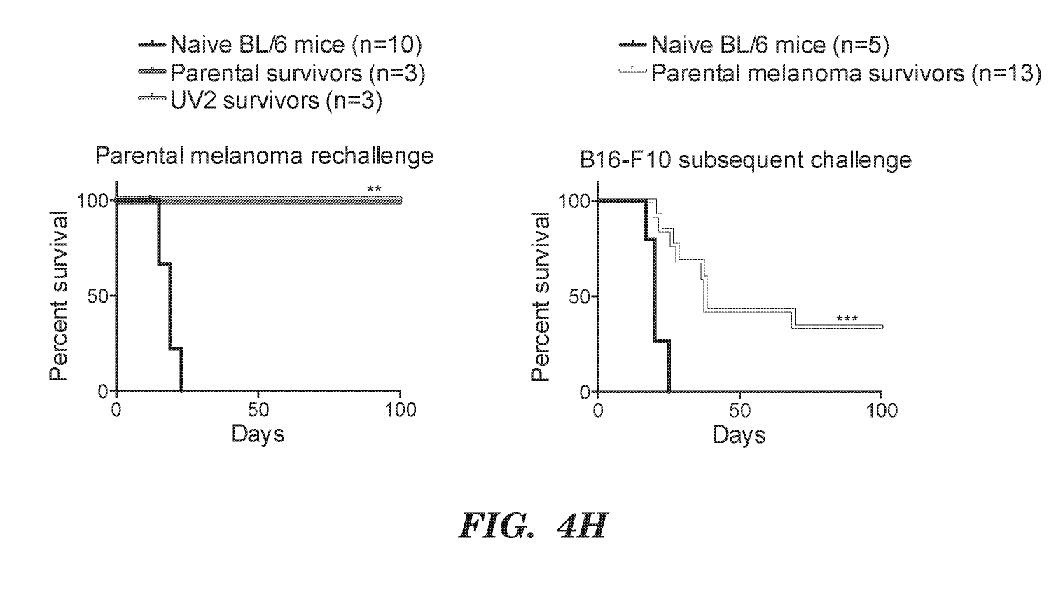

To examine the mechanism by which addition of imiquimod and aFP promotes anti-tumor responses in the neoantigen-deficient context, gene expression was compared in treated parental melanomas by RNA-sequencing. In melanomas treated with triple therapy compared to anti-PD-1 alone, GSEA identified significant enrichment of several immune-related KEGG gene sets (FIG. 4A, Table 2). In parental melanomas, addition of imiquimod and aFP to checkpoint inhibitor antibodies substantially increased intratumoral CD3+ T cell density and CD8:Treg ratio compared to isotype-control antibodies (FIGS. 4B, 4C). Imiquimod alone or with anti-PD-1 or aFP expanded the granzyme B+ fraction of CD8+ TILs (FIG. 4C). In draining lymph nodes (dLNs), programmed cell death 1 ligand 2 (PD-L2) expression on CD11c+ dendritic cells (DCs) was reduced by imiquimod, indicating imiquimod makes DCs less suppressive (FIG. 4C). Notably, similar changes were observed in both directly-treated and contralateral (untreated) tumors and dLNs, indicating that local imiquimod has broad immune effects (FIG. 4A). These data indicate that imiquimod enhances antigen presentation and activates the T cell compartment independently of anti-PD-1 or anti-CTLA-4, but checkpoint blockade is needed to increase T cell infiltration into tumors.

Depletion of CD8+ T cells abrogated triple therapy-mediated parental melanoma regression and survival, confirming their critical role for therapeutic benefit (FIG. 4D). However, no measurable changes in TCR repertoire richness, diversity, or clonality were detected between isotype-matched control, anti-PD-1, and triple therapy groups (FIG. 4E), indicating that early efficacy of triple therapy is not due to oligoclonal T cell expansion or recruitment of more unique T cell clones. Instead, triple therapy can lead to polyclonal T cell expansion or enhanced priming, quality, or function of antigen specific CD8+ T cells.

To examine features of human melanomas with low neoantigen burdens but successful responses to checkpoint blockade, a dataset of pre-treatment melanoma biopsies from patients receiving ipilimumab was interrogatedll. Patients with low neoantigen loads were categorized as ipilimumab responders or non-responders as described herein in the Methods section. GSEA of RNA-sequencing profiles revealed significantly higher expression of genes associated with IFN-α and IFN-γ signaling in responders than non-responders (Table 3). This likely reflects greater type I interferon signaling in pre-treatment tumors that is associated with spontaneous tumor inflammation34-37. The top GO biological process gene sets enriched in low neoantigen responders were pigmentation-related (Table 3), and the overarching GO pigmentation gene set (GO:0043473) was also significantly enriched in responders (FIG. 4F). The same GO pigmentation gene set was enriched in parental mouse melanomas following triple therapy but not anti-PD-1 monotherapy, and triple therapy is also associated with increased IFN-α response and IFN-γ response (Table 2, FIG. 4F). This indicates that addition of aFP and imiquimod mediates changes in tumor gene expression profiles that partially recapitulate the differences between human low neoantigen responders versus non-responders.

The GO pigmentation gene set includes melanocyte differentiation antigens such as gp100 and tyrosinase. Without wishing to be bound by theory this indicates that upregulated wildtype melanocytic antigens can be targets of T cells following triple therapy. Therefore, mouse parental melanomas were analyzed for melanocytic antigen recognition by tumor-infiltrating T cells using gp100:H-2Db tetramer staining. Triple therapy produced a strong induction of gp100-tetramer-positive CD8+ TILs (p<0.001) as compared to either no treatment or anti-PD-1 alone (FIG. 4G). Thus, addition of imiquimod and aFP leads to measurable expansion of CD8+ T cell populations capable of recognizing wildtype melanocytic antigens within anti-PD-1-treated melanomas.

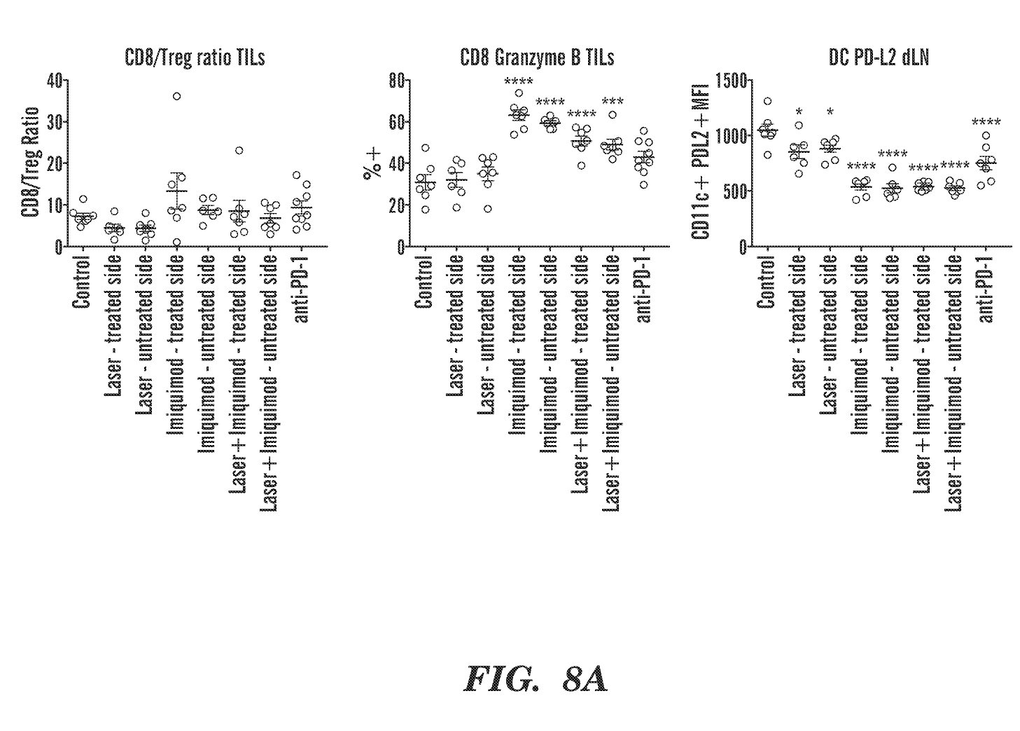

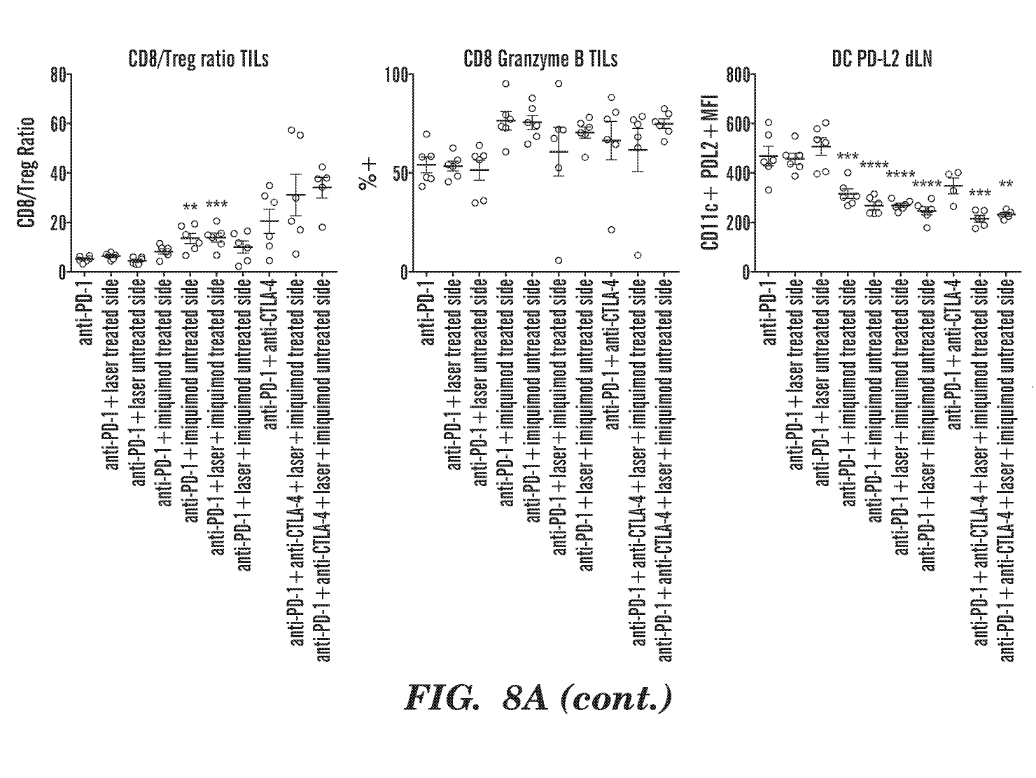

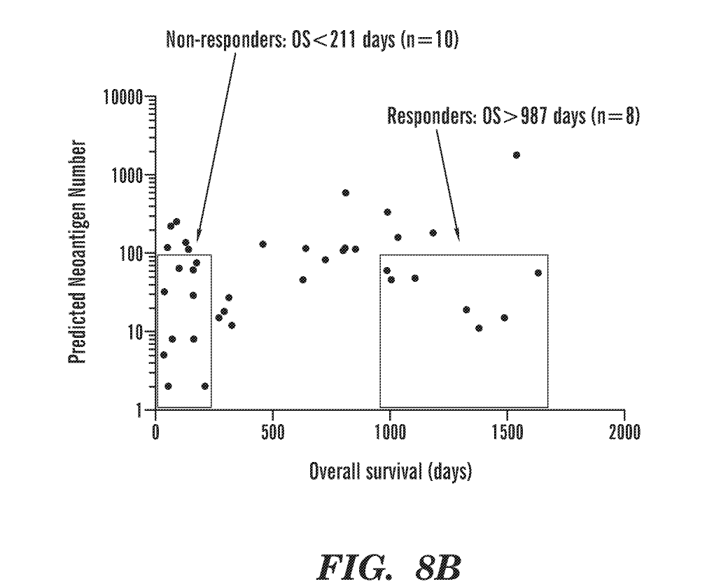

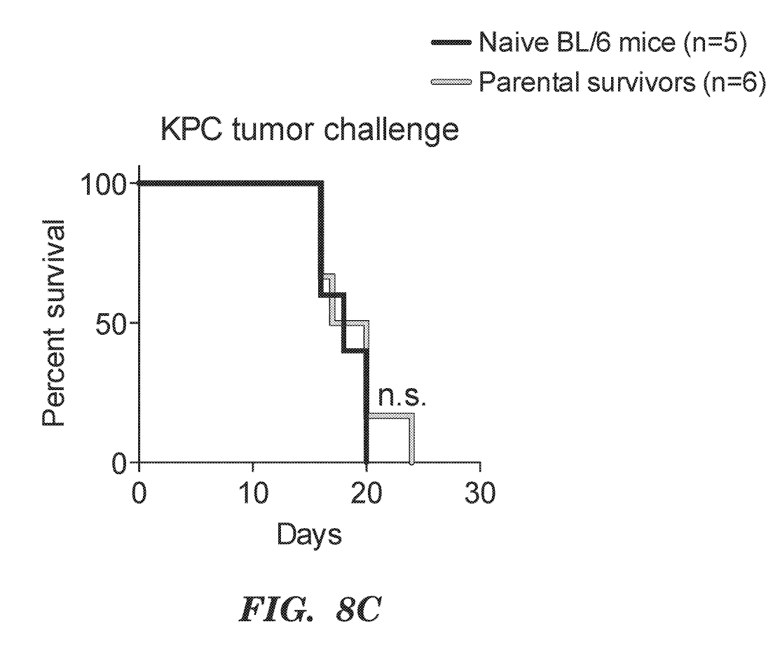

Finally, to evaluate long-term immunity, mice with complete melanoma regressions after triple therapy were rechallenged with a second melanoma inoculation (FIG. 4H). Unexpectedly, 3 of 3 UV2 melanoma (neoantigen-expressing) survivors had memory responses that mediated rejection of parental (neoantigen-deficient) melanomas. Thus, while addition of mutations was sufficient to provoke a stronger anti-melanoma immune response (FIG. 1C), long-term responses after triple therapy were not restricted to putative neoantigens. In addition, 30% of parental melanoma survivors after combination therapy were protected against the unrelated B16-F10 mouse melanoma (FIG. 4H). In contrast, parental melanoma survivors had no immunity against KPC pancreatic tumors (FIG. 8C). Consistent with increased frequency of gp100-recognizing CD8+ T cells (FIG. 4G), these results indicate that there is long-term immune recognition of shared-lineage tumor epitopes not restricted to neoantigens in successful responders to combination immunotherapy.

Taken together, these results demonstrate two mechanisms by which cancer responses to immune checkpoint blockade can be enhanced: introduction of neoantigens and addition of aFP and imiquimod. Induction of UVB-associated mutations in the anti-PD-1-resistant BRAF(V600E)/Pten−/− melanoma mode125 was sufficient to overcome resistance to checkpoint blockade. In contrast to poorly immunogenic parental tumors, mutagenized UV2 melanomas were characterized by accumulation of dysfunctional T cells that were reinvigorated by anti-PD-1, resulting in complete tumor regressions and long-term survival. These findings validate the functional importance of high mutational loads observed in human cancers38,39.

For cancers bearing low mutational burdens, a novel therapeutic strategy was investigated, by which responses to checkpoint blockade can be achieved. Addition of imiquimod and aFP to checkpoint blockade enhances inflammation in melanoma and pancreatic adenocarcinoma, with innate immune activation and increased CD8+ T cell function leading to systemic complete responses. Of note, triple therapy produced changes in gene expression paralleling the elevated IFN signaling and pigment-related transcript levels observed in human pre-treatment melanoma biopsies from ipilimumab responders among a low neoantigen subset of patients.

Vitiligo is associated with clinical efficacy of PD-1 blockadel3 and is a treatment related side effect in patients with melanoma but not other cancers¹⁻³. Vitiligo is unlikely to result from immune responses against neoantigens, which are randomly distributed by UVR and unlikely to be shared among patches of cutaneous melanocytes. Instead, autoimmune destruction of melanocytes could arise from responses against wildtype antigens shared by normal melanocytes and melanoma cells. The melanoma-bearing mice in this study did not develop obvious vitiligo or leukotrichia, but still exhibited evidence of epitope spreading to melanocytic antigens, with induction of CD8+ T cells recognizing gp100, abscopal tumor regressions, and long-term immunity against unrelated melanomas. It is possible that such epitope spreading to wildtype melanocytic antigens occurs in human melanoma patients and contributes to immunotherapy efficacy even in individuals without overt vitiligo. Indeed, a significant fraction of melanoma patients who respond to anti-PD-1 do not develop vitiligo. These data, with the lack of vitiligo or pancreatitis in melanoma and pancreatic adenocarcinoma models, indicate that there is a therapeutic window in which combinations like imiquimod+aFP+immune checkpoint blockade can drive responses against tumor lineage self-antigens and have clinical benefit without dangerous toxicities involving autoimmune destruction of the organ of tumor origin. Thus, such therapeutic strategies can be used to safely achieve significant efficacy in non-inflamed cancers that are refractory to checkpoint inhibitors in the clinic.

Methods

Cell lines and tissue culture. KPC was a gift from Stephanie Dougan and B16-F10 was purchased from ATCC. The D4M.3A.3 (“parental”) cell line was derived from single cell cloning of D4M.3A. To generate the D3UV2 (“UV2”) and D3UV3 (“UV3”) cell lines, D4M.3A.3 cells were sequentially irradiated in vitro with 25 mJ/cm2 UVB 3 times before isolating and culturing single cell clones from the surviving population. All cell lines were cultured in DMEM supplemented with 10% fetal bovine serum.

Cell viability assay. Melanoma cells were counted in trypan blue and plated at 4,000 viable cells per well onto 96-well plates. After 16 hrs of serum starvation, cells were rescued with DMEM containing 10% FBS (day 0). CellTiter-Glo™ Cell Viability assay kit (Promega™) luminescence was measured on days 0, 1, 2, and 3 according to the manufacturer's instructions.

Cell counting. Melanoma cells were counted in Trypan blue and plated at 12,500 viable cells per well onto 24-well plates in DMEM containing 0.5% FBS. After 16 hrs of serum starvation, cells were rescued with DMEM containing 10% FBS (day 0). Total numbers of viable cells per well was counted on days 0, 1, 2, and 3.

Whole-exome sequencing. DNA from melanoma cell lines was extracted using the Gentra Puregene™ Cell Kit (Qiagen™) according to manufacturer's instructions. Whole exome sequencing was performed using the Agilent™ whole exome capture kit (SureSelect™ Mouse All Exon). Captured material was indexed and sequenced on the Illumina™ platform at the Wellcome Trust Sanger Institute™. Raw pair end sequencing reads were aligned with BWA-MEM to the GRCm38 mouse reference genome 1. The SAMTools™ Mpileup™ multi sample variant calling approach was used to simultaneously detect variants from aligned sequencing data of parental and derived lines. De novo variants in the derived lines were then detected by excluding variants co-occurring with the parental lines. These de novo variants were further refined by removing low quality variants and germline variants identified by the Mouse Genome variation Project2.

In vivo mouse studies. 8-week-old female C57BL/6 and NSG mice were obtained from Jackson Laboratory™ (Bar Harbor, Me.). To minimize variation in pathogen exposure in these experiments, all mice were obtained from the same mouse facility at the same age and housed together. Melanoma cells (1×106 cells per site in PBS) were inoculated subcutaneously at the flanks. Blocking antibodies were administered intraperitoneally at a dose of 200 ug per mouse. For UV clone experiments, antibodies were administered on days 8, 10, 12, 14, and 16 after tumor cell inoculation. anti-PD-1 (29F.1A12) was a gift from Gordon Freeman and isotype-matched (2A3) antibodies were acquired from BioXCell™. For combination therapy experiments, anti-PD-1 (29F.1A12) or isotype matched (2A3) and anti-CTLA-4 (9D9) or isotype-matched (MPC-11) were administered on days 6, 8, and 10 (triple therapy) or on days 8, 10, and 12 (quadruple therapy). Left flank tumors were treated with 5% imiquimod (Strides Pharma™) or vehicle lotion concurrently with antibody treatments, and aFP using a CO2 laser (UltraPulse DeepFX™, Lumenis™, Yokneam, Israel) on the first and last day of antibody treatment. For aFP, a 5 mm×5 mm scanning pattern with 100 mJ energy per pulse, 5% coverage, and 120 um nominal spot size was applied. AFP dosimetry: 100 mJ energy per pulse penetrates to −2.5 mm depth below the skin surface, thus assuming a 50 mm3 tumor extends from about 0.3 mm from the skin surface (estimate based on Hansen et al, Anat Rec 210:569-573, 1984), a 5×5 mm aFP pattern provides 100% tumor coverage and reaches ˜2.4% of the tumor volume (5%×[23.5/50 mm3]). For rechallenge experiments, mice were inoculated with 1×105 cells at one flank. For CD8 depletion, rat anti-mouse CD8a (clone 2.43) or isotype-matched (LTF-2) antibody was administered every 3 days for the duration of the experiment, starting 6 days before tumor inoculation. Tumor volume was calculated from caliper measurements as length×(width2/2). For experiments evaluating survival, mice were sacrificed when tumors reached a maximum volume of 4000 mm3 or 500 mm3 in experiments with one or two tumors per mouse, respectively. All studies and procedures involving animal subjects were performed in accordance with policies and protocols approved by the Institutional Animal Care and Use Committee at Massachusetts General Hospital.

Survival and tumor response analysis. Kaplan-Meier analysis was conducted using the log-rank (Mantel-Cox) test. p values less than 0.05 were considered statistically significant.

Immunohistochemical analyses. Mouse tumors were collected 5 days after treatment initiation and formalin-fixed paraffin-embedded (FFPE). Slides were baked for 60 minutes in a 60° C. oven and loaded into the Bond III™ staining platform. Slides were antigen retrieved in Bond™ Epitope Retrieval 1 for 30 minutes at 100° C. then incubated with CD3 (Abcam™, ab16669) at 1:150 diluted in Bond™ Primary Antibody diluent for 30 minutes at room temperature. Primary antibody was detected using Bond™ Polymer Refine Detection kit, slides were developed in DAB, then dehydrated and coverslipped. For each of 3 samples per group, 3 random 20× magnification fields were chosen at the tumor center for quantification. The open-source CellProfiler™ cell image analysis software3 was used to quantify positively stained cells in each image. The analysis pipeline utilized the UnmixColors module to separate each image into one of the Hematoxylin stain and one of the DAB stain. The EnhanceOrSuppressFeatures module was applied to the DAB image to enhance cellular features. Finally, the IdentifyPrimaryObjects module was used to count the number of cells present in the enhanced image.

Immunofluorescence analyses. Mouse tumors were collected 5 days after treatment initiation, fixed in 4% PFA at room temperature for 4 hours followed by submersion in 30% sucrose overnight at 4° C., embedded in OCT, and sectioned into 10 micrometer sections on a New England Biomedical Services™ HM505E cryostat. For fixation and permeabilization, samples were subjected to one of the following: (1) acetone submersion for 5 minutes at room temperature, (2) submersion in 4% PFA for 10 minutes at room temperature followed by submersion in 0.2% Triton solution for 3 minutes at room temperature, (3) submersion in the eBioscience™ Foxp3 fixation/permeabilization reagent for 20 minutes at room temperature. Samples were then washed with 2% BSA 0.02% Tween solution and blocked with 2% BSA solution for 5 minutes at room temperature. Samples were stained at room temperature for 1 hour in 2% BSA solution or Foxp3 Fix Perm Kit permeabilization buffer and washed 2 times in PBS solution. Samples were imaged on a Leica™ Confocal Microscope.

Statistical analysis. Statistical analyses were performed using GraphPad™ Prism™ Significance was determined by two-tailed Student's t tests for two-way comparisons and ANOVA with Tukey's method or Dunnett's method for multiple comparisons. p values less than 0.05 were considered statistically significant.

Flow cytometry. Upon sacrifice, tumor and inguinal (draining) lymph node were isolated and weighed dry. Both were mechanically disaggregated in collagenase type I (400U/ml; Worthington Biochemical™), and then placed on a shaker at 37° C. for 30 minutes. Digests were smashed through 70 um filters to generate a single-cell suspension. For tumors, a Percoll™ gradient ( 40/70%, GE Healthcare™) was used to enrich for leukocytes (TILs). TILs and dLN cells were resuspended in buffer (PBS with 1% FCS and 2 mM EDTA). For tetramer assays, cells were first stained with APC-conjugated H-2Db gp100 tetramer EGSRNQDWL (MBL™ International). Samples were then stained with combinations of the following fluorescently-conjugated antibodies (BioLegend™): anti-CD45.2 (104), anti-CD3c (145-2c11), anti-CD8a (53-6.7), anti-CD4 (RM4-5), anti-CD11b (M1/70), anti-CD11c (N418), anti-I-A/I-E (M5/114.15.2), anti-PD-L1 (CD274;10F.9G2), anti-PD-L2 (CD273; TY25), anti-B7-1 (CD80; 16-10A1), anti-B7-2 (CD86;GL-1), anti-CD40 (HB14), anti-CD44 (BJ18), and anti-PD-1 (RMP1-30). For intracellular staining, cells were fixed and permeabilized using the FoxP3 Transcription Factor Staining Kit (eBioscience™) after surface staining and stained with the following fluorescently conjugated antibodies: anti-FoxP3 (FJK-16s; ebioscience), anti-Ki67 (B56; BD Biosciences™) and anti-Granzyme B (GB11; BioLegend™). Flow cytometry data were acquired on the BD™ LSRII flow cytometer and analyzed using FlowJo™ software (Tree Star™).

TCR deep sequencing and clonotype diversity analysis. Subcutaneous mouse melanoma grafts were collected 11 days after tumor cell inoculation in C57BL/6 mice. anti-PD-1 or isotype control treatments were initiated 5 days prior to sample collection. DNA was extracted and sequenced by Adaptive Biotechnologies™ using “survey” sequencing depth. Entropy was calculated by summing the frequency of each clone times the log (base 2) of the same frequency over all rearrangements in a sample. Clonality was calculated by normalizing entropy using the total number of unique rearrangements and subtracting the result from 1.

Analysis of TCGA melanomas. Survival analysis based on expression-based patient stratification was conducted using the UZH™ Cancer Browser″.

RNA-sequencing of bulk mouse tumors. Total RNA was isolated and purified from mouse melanomas 11 days after tumor cell inoculation using the TissueLyser™ II and RNeasy™ extraction kit (Qiagen™). 76 bp paired-end sequencing was performed on an Illumina™ HiSeq2500 instrument using the TruSeq™ RNA Sample Preparation Kit v2. Libraries were sequenced to an average depth of 15.5 million paired-end reads of length 76 bp. The reads were mapped to the UCSC™ mouse transcriptome (genome build mm10) using Bowtie™ 25 and expression levels of all genes were quantified using RSEM6. On average 79.8% of the reads mapped to the transcriptome in each sample (range 78.4-81.6%). RSEM yielded an expression matrix (genes×samples) of inferred gene counts, which was converted to TPM (transcripts per million).

Determining differentially expressed genes and enriched gene sets. Normalized RNA-sequencing data were filtered to remove genes with an average TPM of less than 1. Gene set enrichment analysis was performed using GSEA software (Broad Institute of Harvard and MIT™) with default settings. KEGG, GO terms (biological process and molecular function), and Hallmark gene set databases were evaluated. GSEA statistics were assessed by 1000 iterations of the gene set permutations. Differential gene expression was estimated using the DESeq2 R package.

Analysis of human melanoma gene expression. A previously published dataset of melanoma patients treated with ipilimumab included 40 melanoma patients with both whole-exome sequencing and RNA-sequencing7. In the present study, the low neoantigen subset of patients was defined to include those with fewer than 100 predicted neoantigens with <50 nM binding affinity for HLA class I molecules. Of these, 8 patients were categorized as ipilimumab responders (overall survival>987 days) and 10 patients as non-responders (overall survival<211 days) (FIG. 8B). Low neoantigen responders were compared to non-responders by GSEA as described above.

### REFERENCES

1. Hodi, F. S. et al. Improved survival with ipilimumab in patients with metastatic melanoma. N. Engl. J. Med. 363, 711-723 (2010).
2. Topalian, S. L. et al. Safety, activity, and immune correlates of anti-PD-1 antibody in cancer. N. Engl. J. Med. 366, 2443-2454 (2012).
3. Hamid, O. et al. Safety and Tumor Responses with Lambrolizumab (Anti-PD-1) in Melanoma. N. Engl. J. Med. (2013). doi:10.1056/NEJMoa1305133
4. Sharma, P. & Allison, J. P. Immune Checkpoint Targeting in Cancer Therapy: Toward Combination Strategies with Curative Potential. Cell 161, 205-214 (2015).
5. Ji, R.-R. et al. An immune-active tumor microenvironment favors clinical response to ipilimumab. Cancer Immunol. Immunother. 61, 1019-1031 (2012).
6. Tumeh, P. C. et al. PD-1 blockade induces responses by inhibiting adaptive immune resistance. Nature 515, 568-571 (2014).
7. Herbst, R. S. et al. Predictive correlates of response to the anti-PD-L1 antibody MPDL3280A in cancer patients. Nature 515, 563-567 (2014).
8. Snyder, A. et al. Genetic Basis for Clinical Response to CTLA-4 Blockade in Melanoma. N. Engl. J. Med. 141119140020009 (2014). doi:10.1056/NEJMoa1406498
9. Rizvi, N. A. et al. Cancer immunology. Mutational landscape determines sensitivity to PD-1 blockade in non-small cell lung cancer. Science 348, 124-128 (2015).
10. Le, D. T. et al. PD-1 Blockade in Tumors with Mismatch-Repair Deficiency. N. Engl. J. Med. 372, 2509-2520 (2015).
11. Van Allen, E. M. et al. Genomic correlates of response to CTLA-4 blockade in metastatic melanoma. Science 350, 207-211 (2015).
12. Hugo, W. et al. Genomic and Transcriptomic Features of Response to Anti-PD-1 Therapy in Metastatic Melanoma. Cell 165, 35-44 (2016).
13. Hua, C. et al. Association of Vitiligo With Tumor Response in Patients With Metastatic Melanoma Treated With Pembrolizumab. JAMA Dermatol 152, 45-51 (2016).
14. Manstein, D., Herron, G. S., Sink, R. K., Tanner, H. & Anderson, R. Fractional photothermolysis: a new concept for cutaneous remodeling using microscopic patterns of thermal injury. Lasers Surg. Med. 34, 426-438 (2004).
15. Gubin, M. M. et al. Checkpoint blockade cancer immunotherapy targets tumour specific mutant antigens. Nature 515, 577-581 (2014).
16. van Rooij, N. et al. Tumor exome analysis reveals neoantigen-specific T-cell reactivity in an ipilimumab-responsive melanoma. J. Clin. Oncol. 31, e439-42 (2013).
17. Robbins, P. F. et al. Mining exomic sequencing data to identify mutated antigens recognized by adoptively transferred tumor-reactive T cells. Nat Genet 19, 747-752 (2013).
18. Linnemann, C. et al. High-throughput epitope discovery reveals frequent recognition of neo-antigens by CD4+ T cells in human melanoma. Nat Med 21, 81-85 (2014).
19. Cohen, C. J. et al. Isolation of neoantigen-specific T cells from tumor and peripheral lymphocytes. J. Clin. Invest. 125, 3981-3991 (2015).
20. Gros, A. et al. Prospective identification of neoantigen-specific lymphocytes in the peripheral blood of melanoma patients. Nat Med 22, 433-438 (2016).
21. Tran, E. et al. Cancer immunotherapy based on mutation-specific CD4+ T cells in a patient with epithelial cancer. Science 344, 641-645 (2014).
22. Vetizou, M. et al. Anticancer immunotherapy by CTLA-4 blockade relies on the gut microbiota. Science 350, 1079-1084 (2015).
23. Sivan, A. et al. Commensal Bifidobacterium promotes antitumor immunity and facilitates anti-PD-L1 efficacy. Science 350, 1084-1089 (2015).
24. Jenkins, M. H. et al. Multiple murine BRafV600E melanoma cell lines with sensitivity to PLX4032. Pigment Cell Melanoma Res (2014).
25. Dankort, D. et al. Braf(V600E) cooperates with Pten loss to induce metastatic melanoma. Nat Genet 41, 544-552 (2009).
26. Lawrence, M. S. et al. Mutational heterogeneity in cancer and the search for new cancer-associated genes. Nature 499, 214-218 (2013).
27. Subramanian, A. & Tamayo, P. Gene set enrichment analysis: A knowledge based approach for interpreting genome-wide expression profiles. (2005).
28. Rooney, M. S., Shukla, S. A., Wu, C. J., Getz, G. & Hacohen, N. Molecular and genetic properties of tumors associated with local immune cytolytic activity. Cell 160, 48-61 (2015).
29. Naylor, M. F. et al. Treatment of lentigo maligna with topical imiquimod. Br. J. Dermatol. 149 Supp! 66, 66-70 (2003).
30. Swetter, S. M., Chen, F. W., Kim, D. D. & Egbert, B. M. Imiquimod 5% cream as primary or adjuvant therapy for melanoma in situ, lentigo maligna type. Journal of the American Academy of Dermatology 72, 1047-1053 (2015).
31. Kawakubo, M. et al. Fractional Laser Exposure Can Induce Neutrophil-mediated Tumor Destruction and Stimulate Systemic Anti-tumor Immune Response. In submission (2016).
32. Postow, M. A. et al. Nivolumab and Ipilimumab versus Ipilimumab in Untreated Melanoma. N. Engl. J. Med. 372, 2006-2017 (2015).
33. Larkin, J. et al. Combined Nivolumab and Ipilimumab or Monotherapy in Untreated Melanoma. N. Engl. J. Med. 373, 23-34 (2015).
34. Fuertes, M. B. et al. Host type I IFN signals are required for antitumor CD8+ T cell responses through CD8α+ dendritic cells. J. Exp. Med. 208, 2005-2016 (2011).
35. Diamond, M. S. et al. Type I interferon is selectively required by dendritic cells for immune rejection of tumors. J. Exp. Med. 208, 1989-2003 (2011).
36. Spranger, S. et al. Up-Regulation of PD-L1, IDO, and Tregs in the Melanoma Tumor Microenvironment Is Driven by CD8+ T Cells. Science Translational Medicine 5, 200ra116-200ra116 (2013).
37. Bald, T. et al. Immune cell-poor melanomas benefit from PD-1 blockade after targeted type I IFN activation. Cancer Discov 4, 674-687 (2014).
38. Lawrence, M. S. et al. Discovery and saturation analysis of cancer genes across 21 tumour types. Nature 505, 495-501 (2014).
39. Schumacher, T. N. & Schreiber, R. D. Neoantigens in cancer immunotherapy. Science 348, 69-74 (2015).

### References for Methods

1. Li, H. Aligning sequence reads, clone sequences and assembly contigs with BWA MEM. arXiv (2013).
2. Keane, T. M. et al. Mouse genomic variation and its effect on phenotypes and gene regulation. Nature 477, 289-294 (2011).
3. Carpenter, A. E. et al. CellProfiler: image analysis software for identifying and quantifying cell phenotypes. Genome Biol 7, R100 (2006).
4. Cheng, P. F., Dummer, R. & Levesque, M. P. Data mining The Cancer Genome Atlas in the era of precision cancer medicine. Swiss Med Wkly 145, w14183 (2015).
5. Langmead, B. & Salzberg, S. L. Fast gapped-read alignment with Bowtie 2. Nat Meth 9, 357-359 (2012).
6. Li, B. & Dewey, C. N. RSEM: accurate transcript quantification from RNA-Seq data with or without a reference genome. BMC Bioinformatics 12, 323 (2011).
7. Van Allen, E. M. et al. Genomic correlates of response to CTLA-4 blockade in metastatic melanoma. Science 350, 207-211 (2015).

1. A method for treating cancer in a subject, the method comprising:
(a) administering at least one drug to a subject having a tumor, and
(b) contacting tissue of the tumor with a fractional laser,
thereby treating cancer in the subject.

2. (canceled)

3. The method of claim 1, wherein the at least one drug is an immune checkpoint inhibitor.

4. The method of claim 3, wherein the immune checkpoint inhibitor is an inhibitor of PD1, PDL1, TIM-3, or CTLA4.

5. The method of claim 3, wherein the immune checkpoint inhibitor is ipilimumab, tremelimumab, nivolumab, or pembrolizumab.

6. (canceled)

7. (canceled)

8. The method of claim 1, wherein the at least one drug is an agonist of TLR3, TLR7, TLR8 or TLR9.

9. The method of claim 8, wherein the TLR7 agonist is imiquimod, reiquimod, or gardiquimod.

10.-15. (canceled)

16. The method of claim 1, wherein the cancer is melanoma or pancreatic cancer.

17. The method of claim 1, wherein the fractional laser is a CO2 laser.

18. The method of claim 1, wherein the fractional laser penetrates to a depth of at least 0.1 mm into the tumor tissue.

19. (canceled)

20. The method of claim 1, wherein treatment with the fractional laser does not damage the stratum corneum.

21. The method of claim 1, wherein treatment with the fractional laser does not induce scarring or crusting of the tumor tissue.

22. The method of claim 1, wherein the area of treatment comprises at least 0.25 mm².

23. The method of claim 1, wherein the energy of the fractional laser is 1 mJ to 200 mJ.

24. The method of claim 23, wherein 50 mJ of energy is used for a superficial lesion and 200 mJ of energy is used for a deep tumor.

25. The method of claim 1, wherein the pulse duration of the fractional laser is 100 usec to 10 msec.

26. The method of claim 25, wherein the pulse duration of the fractional laser is 2 msec.

27. The method of claim 1, wherein the spot size of the fractional laser is 10 um to 1 mm.

28. The method of claim 1, wherein the penetration depth of the fractional laser is ⅓ the depth of the tumor.

29.-57. (canceled)

58. A method for treating cancer in a subject, the method comprising:
(a) administering at least one drug to a subject having a tumor, and
(b) contacting tissue of the tumor with radiofrequency (RF) energy, thereby treating cancer in the subject.

59. A method for treating cancer in a subject, the method comprising:
(a) administering at least one drug to a subject having a tumor, and
(b) contacting tissue of the tumor to form microscopic treatment zones (MTZs), thereby treating cancer in the subject.


SEQUENCE LISTING (1 sequence)

SEQ ID NO: 1 - protein, 9 aa, Artificial Sequence
  Description of Artificial Sequence Synthetic peptide
EGSRNQDWL